(19) United States
(12) Patent Application Publication
(10) Pub. No.: US 2024/0216509 A1
(43) Pub. Date: Jul. 4, 2024

(54) CHIMERIC POLYPEPTIDES, NUCLEIC ACID MOLECULES, CELLS, AND RELATED METHODS

(71) Applicant: CHILDREN'S HOSPITAL MEDICAL CENTER, Cincinnati, OH (US)

(72) Inventors: Stephen WAGGONER, Cincinnati, OH (US); Hermine BRUNNER, Cincinnati, OH (US); Seth REIGHARD, Cincinnati, OH (US)

(73) Assignee: CHILDREN'S HOSPITAL MEDICAL CENTER, Cincinnati, OH (US)

(21) Appl. No.: 17/052,894

(22) PCT Filed: May 6, 2019
(86) PCT No.: PCT/US2019/030803

§ 371 Date: Nov. 4, 2020

Related U.S. Application Data
(60) Provisional application No. 62/667,669, filed on May 7, 2018.

(51) Int. Cl. A61K 39/00; A61K 38/00; C07K 14/705; C07K 14/725; C07K 14/73; C12N 5/0783; C12N 15/86

(52) U.S. Cl. CPC A61K 39/4631; A61K 39/4611; A61K 39/4613; C07K 14/70503; C07K 14/7051; C07K 14/70514; C07K 14/70517; C07K 14/70521; C07K 14/70532; C07K 14/70575; C07K 14/70578; C07K 14/70596; C12N 5/0636; C12N 5/0646; C12N 15/86; A61K 38/00; C07K 2319/02; C07K 2319/03; C12N 2740/15043

(57) ABSTRACT

Some embodiments of the invention include chimeric polypeptides. Other embodiments of the invention include chimeric nucleic acid molecules encoding a chimeric polypeptide. Other embodiments of the invention include vectors comprising a chimeric nucleic acid molecule. Still other embodiments of the invention include cells comprising a chimeric nucleic acid molecule, a chimeric polypeptide, or both. Yet other embodiments of the invention include methods of making cells. Some embodiments include methods of treating disease. Additional embodiments of the invention are also discussed herein.

### CROSS-REFERENCE TO RELATED APPLICATIONS

This application is a National Stage Entry of International Application No. PCT/US2019/030803 filed May 6, 2019, entitled “CHIMERIC POLYPEPTIDES, NUCLEIC ACID MOLECULES, CELLS, AND RELATED METHODS” which is herein incorporated by reference in its entirety, and which claims the benefit of U.S. Provisional Application No. 62/667,669, filed May 7, 2018 entitled “Chimeric antigen receptor as a natural killer cell therapy for autoimmune disease” which is herein incorporated by reference in its entirety.

### REFERENCE TO A SEQUENCE LISTING

The instant application contains a Sequence Listing that has been submitted in ASCII format via PatentCenter and is hereby incorporated by reference in its entirety. Said ASCII copy, created on Jul. 5, 2023, is named 2023_07_seq_listing_36821_04054_vFINAL_ST25.txt and is 96,248 bytes in size.

### BACKGROUND

Diseases have plagued humankind for thousands of years. For example, follicular helper T cells (T_{FH}) can be helpful for vaccine and infection elicitation of long-lived humoral immunity, but exaggerated T_{FH} responses can sometimes promote certain diseases, such as autoimmune diseases (e.g., Sjögren's syndrome, juvenile dermatomyositis, multiple sclerosis, type-1 diabetes mellitus, rheumatoid arthritis, and systemic lupus erythematosus (SLE)). Unfortunately, no adequate clinical interventions are currently available for selective depletion of T_{FH} cells to alleviate these diseases. T_{FH} cells also appear to play roles in allergies (e.g., IgE related allergies) and cancer (e.g., lymphoma).

Certain embodiments of the invention address one or more of the deficiencies described above. For example, some embodiments of the invention include chimeric polypeptides. Other embodiments of the invention include chimeric nucleic acid molecules encoding a chimeric polypeptide. Other embodiments of the invention include vectors comprising a chimeric nucleic acid molecule. Still other embodiments of the invention include cells comprising a chimeric nucleic acid molecule, a chimeric polypeptide, or both. Yet other embodiments of the invention include methods of making cells. Some embodiments include methods of treating disease. Additional embodiments of the invention are also discussed herein.

### SUMMARY

Some embodiments of the invention include a chimeric polypeptide wherein the chimeric polypeptide comprises (a) an extracellular segment comprising PD-L1 (Programmed Death-Ligand 1) or a portion of PD-L1, or one or more modifications thereof, (b) a transmembrane segment, and (c) an intracellular segment comprising a first signaling polypeptide and optionally one or more second signaling polypeptides. In some embodiments, the PD-L1 is a mammalian PD-L1, a mouse PD-L1, a dog PD-L1, a cat PD-L1, a rat PD-L1, a pig PD-L1, a woodchuck PD-L1, a cow PD-L1, a monkey PD-L1, a cynomolgus monkey PD-L1, a primate PD-L1, a human PD-L1, or SEQ ID NO:38. In other embodiments, the extracellular segment comprises SEQ ID NO: 14 or SEQ ID NO:15, or one or more modifications thereof. In still other embodiments, the extracellular segment further comprises one or more spacer sequences, where each of the one or more spacer sequences comprises from 5 amino acids to about 1000 amino acids. In yet other embodiments, the extracellular segment further comprises one or more spacer sequences and the one or more of the spacer sequences comprises CD8α, a portion of CD8α, a hinge CD8α sequence, a leader CD8α sequence, CD8β, a portion of CD8β, CD4, a portion of CD4, CD28, or a portion of CD28, or one or more modifications thereof, or combinations thereof. In certain embodiments, the extracellular segment comprises one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, or a portion thereof, or one or more modifications thereof. In some embodiments, the transmembrane segment comprises SEQ ID NO:16, SEQ ID NO:17, or portions thereof, or one or more modifications thereof. In other embodiments, the first signaling polypeptide is CD3ζ, FcRγ, FcRβ, CD3γ, CD3δ, CD3ε, CD5, CD22, CD79a, CD79b, or CD66d, or portions thereof, or one or more modifications thereof. In still other embodiments, the first signaling polypeptide comprises SEQ ID NO:18, SEQ ID NO:19, or SEQ ID NO:20, or one or more modifications thereof. In yet other embodiments, the intracellular segment comprises a second signaling polypeptide and the second signaling polypeptide is CD2, CD4, CD5, CD8α, CD8β, CD28, CD134, CD137, ICOS, or CD154, or portions thereof, or one or more modifications thereof. In certain embodiments, the intracellular segment comprises a second signaling polypeptide and the second signaling polypeptide comprises SEQ ID NO:21 or SEQ ID NO:22, or one or more modifications thereof. In some embodiments, the intracellular segment comprises SEQ ID NO:18, SEQ ID NO:19, SEQ ID NO:20, SEQ ID NO:21, SEQ ID NO:22, SEQ ID NO:23, SEQ ID NO:24, or one or more modifications thereof. In other embodiments, the chimeric polypeptide comprises one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:38, or one or more modifications thereof. In still other embodiments, the chimeric polypeptide comprises SEQ ID NO:25, SEQ ID NO:26, SEQ ID NO:27, SEQ ID NO:28, SEQ ID NO:29, SEQ ID NO:30, SEQ ID NO:31, or one or more modifications thereof.

Some embodiments of the invention include a chimeric nucleic acid molecule encoding the chimeric polypeptide as disclosed herein. In some embodiments, the chimeric nucleic acid molecule has at least an 80% identity to SEQ ID NO:32. In other embodiments, the chimeric nucleic acid molecule has at least an 90% identity to SEQ ID NO:32. In yet other embodiments, the chimeric nucleic acid molecule encoding the polypeptide is SEQ ID NO: 32. In still other embodiments, the chimeric nucleic acid molecule is in a cell, an insect cell, a mammalian cell, a human cell, an sf9 insect cell, a rat cell, a mouse cell, a CHO cell, an immune cell, an immune cell progenitor, a T cell, a T cell progenitor, an NK cell, KHYG1, NK-92, YT, SNK-6, NKL or an NK cell progenitor. In certain embodiments, the chimeric nucleic acid molecule is in an immune cell, an immune cell progenitor, a T cell, a T cell progenitor, an NK cell, KHYG1, NK-92, YT, SNK-6, NKL, or an NK cell progenitor. In some embodiments, the chimeric nucleic acid molecule is in a cell isolated from an animal, a mammal, a primate, or a human. In other embodiments, the chimeric nucleic acid molecule is included in a vector, a viral vector, or a plasmid.

Some embodiments of the invention include a vector comprising any of the chimeric nucleic acid molecules disclosed herein.

Some embodiments of the invention include a cell comprising any chimeric polypeptide disclosed herein, any chimeric nucleic acid molecule disclosed herein, or any vector disclosed herein. In some embodiments, the cell is an immune cell, a stem cell, a mammalian cell, a primate cell, or a human cell. In other embodiments, the cell is autologous or allogeneic. In still other embodiments, the cell is a T cell, a CD8-positive T cell, a CD4-positive T cell, a regulatory T cell, a cytotoxic T cell, or a tumor infiltrating lymphocyte. In yet other embodiments, the cell is an NK cell, an KHYG1 cell, an NK-92 cell, a YT cell, an SNK-6 cell, or an NKL cell.

Some embodiments of the invention include a composition comprising any chimeric polypeptide disclosed herein, any chimeric nucleic acid molecule disclosed herein, any vector disclosed herein, or any cell disclosed herein. In other embodiments, the amount of the chimeric polypeptide, the chimeric nucleic acid molecule, the chimeric vector, or the cell is from about 0.0001% (by weight total composition) to about 99%.

Some embodiments of the invention include a pharmaceutical composition comprising any chimeric polypeptide disclosed herein, any chimeric nucleic acid molecule disclosed herein, any vector disclosed herein, or any cell disclosed herein. In other embodiments, the amount of the chimeric polypeptide, the chimeric nucleic acid molecule, the chimeric vector, or the cell is from about 0.0001% (by weight total composition) to about 50%.

Some embodiments of the invention include a method of producing any cell disclosed herein comprising adding to a cell any chimeric nucleic acid molecule disclosed herein or any vector disclosed herein. Some embodiments of the invention include a method of producing any cell disclosed herein comprising (a) isolating a first cell, (b) adding to the isolated first cell, any chimeric nucleic acid molecule disclosed herein or any vector disclosed herein, to result in any cell disclosed herein, and (c) optionally recovering and/or expanding the resulting cell from step (b). In other embodiments, the first cell is an immune cell, a stem cell, a mammalian cell, a primate cell, or a human cell. In yet other embodiments, the first cell is autologous or allogeneic. In still other embodiments, the first cell is a T cell, a CD8-positive T cell, a CD4-positive T cell, a regulatory T cell, a cytotoxic T cell, or a tumor infiltrating lymphocyte. In certain embodiments, the first cell is an NK cell, an KHYG1 cell, an NK-92 cell, a YT cell, an SNK-6 cell, or an NKL cell.

Some embodiments of the invention include a method for treating an animal with a disease comprising administering any cell as disclosed herein, any cell produced by as disclosed herein, any cell composition as disclosed herein, or any cell pharmaceutical composition as disclosed herein. In some embodiments, the disease is autoimmune disease, allergies, asthma, Sjögren's syndrome, juvenile dermatomyositis, multiple sclerosis, type-1 diabetes mellitus, rheumatoid arthritis, systemic lupus erythematosus (SLE), cancer, blood cancer, solid tumor, leukemia, lymphoma, myeloma, breast cancer, ovarian cancer, glioblastoma, osteosarcoma, medulloblastoma, inflammatory disease, eczema, hepatitis, or an infectious disease. In other embodiments, the disease is autoimmune disease, Sjögren's syndrome, juvenile dermatomyositis, multiple sclerosis, type-1 diabetes mellitus, rheumatoid arthritis, systemic lupus erythematosus (SLE), or cancer. In still other embodiments, the animal is a mammal, primate, human, mouse, or rat. In yet other embodiments, the administration comprises parenteral administration, a mucosal administration, intravenous administration, subcutaneous administration, topical administration, intradermal administration, oral administration, sublingual administration, intranasal administration, or intramuscular administration. In certain embodiments, the animal is in need of treatment.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: PD-1 is a selective marker of human T_{FH} cells (A-C) & Development of an embodiment of PD-L1 CAR NK cells (D-H). (A) Representative PD-1 expression levels determined by flow cytometry on electronically gated subsets of leukocytes in non-inflamed human tonsil. (B) Mean PD-1 expression (geometric MFI) across major leukocytes subsets identified in two independent non-inflamed human tonsils. (C) Mean PD-1 expression (geometric MFI) across major leukocytes subsets identified in three healthy human PBMC. (D) An embodiment of a PD-L1 CAR design—Coding sequence for an example of a CAR construct, color-coded by sequences and separated by [symbol] . (E) An embodiment of a PD-L1 CAR design—Graphical depiction of an example of a CAR protein expressed on cell surface (dashed line), with example sequences. (F) Generation of CAR NK-92 via lentiviral transduction. Percent transduction efficiency of NK-92 with empty lentiviral vector (top-left) and CAR-containing lentiviral vector (bottom-left) as measured by reporter fluorescence. Images of the corresponding sorted fluorescent NK-92 (right, top and bottom) following ˜1 week of culture (growing in characteristic clumps). (G) CAR-transduced NK-92 express CAR-specific mRNA—Graphical depiction of RNA alignment of an embodiment of a CAR-specific qPCR primer/probe sets (top) and the corresponding fold CAR mRNA expression (bottom) in control vs. CAR NK-92. mRNA expression normalized to GAPDH. (H) CAR-transduced NK-92 express surface PD-L1—PD-L1 expression of empty-vector-transduced vs. CAR-lentivirus-transduced NK-92, including PD-L1 fluorescence-minus-one (FMO) control.

FIG. 2: PD-L1 CAR NK cell responses to plate- and cell-associated PD-1. (A) Degranulation (surface exposure of CD107a) of control (left) or CAR (right) NK-92 following a 4-hour incubation in the presence of either anti-PD-L1 IgG vs. goat IgG control (20 μg/mL), or rhPD-1-Fc vs. IgG-Fc control (10 μg/mL). Representative data from one of three experimental replicates is shown. (B) Degranulation (as in A) in response to increasing concentrations of rhPD-1-Fc or IgG1-Fc. Representative data from one of two experimental replicates is shown. (C) Degranulation in the presence of either control or PD-1⁺ drosophila S2 cells (or PMA/Ionomycin) at an effector:target ratio of 1:5 for four hours. Representative data from one of three experimental replicates is shown. (D) Fold degranulation (over control NK-92 with control S2) of CAR NK-92 in the presence of either control, PD-1^{Lo}, or PD-1^{Hi} drosophila S2 cells at a 1:5 E:T for four hours (n=2-3). Dotted line represents average degranulation of control NK-92 cultured with control S2 cells. Data were analyzed via 1-way ANOVA with multiple comparisons. Representative data from one of two experimental replicates is shown. (E) CAR NK-92 degranulation in the presence of control or PD-1⁺ Raji cells for four hours at a 1:5 E:T (n=4). Data analyzed using Student's t-test. Representative data from one of two experimental replicates is shown. (F) Control or PD-1⁺ Raji uptake of propidium iodide following 4-hour co-culture with either control or CAR NK-92 at a 20:1 E:T (n=2-3). Data analyzed using 1-way ANOVA. Representative data from one of two experimental replicates is shown. (G) Generation of target cell lines expressing human PD-1—Contour plots showing PD-1 expression in pAc5/V5-His-PD1-transfected S2 cells, and electronic gates indicating sorting parameters for PD-1⁺ (top left) or PD-1^{Hi} and PD-1^{Lo} (top right) cell populations; resulting PD-1 expression on sorted populations, after 1 week of culture vs. empty-vector-transfected control S2 cells (bottom). (H) Generation of target cell lines expressing human PD-1—Contour plots showing percent PiggyBac transposition efficiency of Raji cells with PB-513 empty-vector (top left) and PD-1-containing PB-513 vector (top right) as measured by reporter fluorescence; resulting PD-1 expression in the fluorescently-sorted Raji cells after 1 week of culture (bottom).

FIG. 3: PD-L1 CAR NK cells selectively kill T_{FH} cells. (A) Representative contour plots of enriched tonsillar CD4 T cells, with circle denoting electronic gating of T_{FH} cells (CXCR5+ PD-1+) following a 4-hour incubation either alone (left, n=1) or in the presence of control NK-92 (middle, n=5) or CAR NK-92 (right, n=5). Percentages indicate mean frequency (±SEM) of all CD4 T cells. (B) Enriched tonsillar CD4 Naïve T, non-T_{FH} memory T, or T_{FH} cell uptake of PI following 4-hour incubation with either control or CAR NK-92 (n=8). Dotted lines represent PI uptake in the absence of NK-92. Data analyzed using Student's t-test. One of two representative experiments is shown. (C) Number of CD4⁺ CD25⁺ CD127^{low} Treg or CD4⁺ PD1⁺ CXCR5⁺ T_{FH} recovered from 4-hour co-cultures of enriched tonsillar CD4 T cells alone or with CAR or non-CAR NK92 cells (n=4). Right panel shows Zombie dye uptake (loss of viability) on electronically gated CD4⁺ CD25⁺ Foxp3⁺ Treg or CD4⁺ PD1⁺ CXCR5⁺ T_{FH} at end of co-culture.

FIG. 4: PD-L1 CAR NK cells kill T cells in T:B co-cultures. (A) Control or CAR NK-92 degranulation following 4-hour co-culture with SEB-stimulated tonsillar T_{FH} and CD27⁺ B cells at an NK:T:B cell ratio of 5:1:2 (n=5). Representative one of three experiments is shown. Data analyzed via Student's t-test. (B) Percent lysis of calcein-AM (CAM)-labeled, SEB-stimulated tonsillar T_{FH} and CD27⁺ B cells after incubation with control or CAR NK-92 for four hours over an increasing E:T ratio, as measured by calcein fluorescence measured in supernatant. Shaded area is ≤percent background lysis of T and B cells alone. Percent lysis calculated as [(sample fluorescence−minimum fluorescence)/(maximum fluorescence−minimum fluorescence)] for 3 biological replicates. (C) T_{FH} and CD27⁺ B cell PI uptake following 4-hour incubation with control or CAR NK-92 (n=4). Uptake in each cell population alone represented by dotted line. Data analyzed via Student's t-test. One of two similar experiments is shown. (D) Frequency of proliferating tonsillar B cells following a 5-day, SEB-stimulated co-culture with CD4 T cells (“No NK-92”) vs. CD4 T cells that were previously cultured with, then depleted of, either control or CAR NK-92 cells. *p<0.05, ***p<0.001, ****p<0.0001. Results analyzed via 1-way ANOVA using Tukey's test for multiple comparisons. (E) ELISA assay optical density (OD) readings at 450 nm comparing levels of immunoglobulin M (IgM) in the supernatant of the aforementioned co-cultures at day 5. Dotted line represents average background readings in blank wells. *p<0.05, ***p<0.001, ****p<0.0001. Results analyzed via unpaired, two-tailed Student's t-test.

FIG. 5: Humanized mouse models of SLE. (A) Splenic PD-1^{high} CD4 T cells (CD19⁻CD3⁺) in humanized NSG mice (n=3) 16 wks after injection of pristane. (B) CD56⁺ ZsGreen⁺ NK in spleen and liver 24 h after injection of 10⁷ CAR cells. (C) Reduced proportions of PD-1^{high} CD4 but not B cells after 3× injections of 5×10⁶ CAR NK (n=2).

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include chimeric polypeptides. Other embodiments of the invention include chimeric nucleic acid molecules encoding a chimeric polypeptide. Other embodiments of the invention include vectors comprising a chimeric nucleic acid molecule. Still other embodiments of the invention include cells comprising a chimeric nucleic acid molecule, a chimeric polypeptide, or both. Yet other embodiments of the invention include methods of making cells. Some embodiments include methods of treating disease. Additional embodiments of the invention are also discussed herein.

As used herein, “wt” or “wild type” refers to a polypeptide or nucleic acid molecule that is naturally found in an organism.

As used herein unless otherwise modified, “one or more modifications” of a polypeptide, a segment of a polypeptide, or a portion of a polypeptide can comprise an insertion, a deletion, a substitution, or combinations thereof. In certain embodiments, one or more modifications can comprise an insertion which can comprise inserting (e.g., at the C-terminus, at the N-terminus, or at another place in the polypeptide or segment or portion thereof) about 1, about 2, about 3, about 4, about 5, about 6, about 7, about 8, about 9, about 10, about 11, about 12, about 13, about 14, about 15, about 16, about 17, about 18, about 19, or about 20 amino acids (e.g., natural amino acids, or modified or unusual amino acids). In certain embodiments, one or more modifications can comprise a deletion which can comprise deleting (e.g., at the C-terminus, at the N-terminus, or at another place in the chimeric polypeptide or segment or portion thereof) about 1, about 2, about 3, about 4, about 5, about 6, about 7, about 8, about 9, about 10, about 11, about 12, about 13, about 14, about 15, about 16, about 17, about 18, about 19, or about 20 amino acids. In certain embodiments, one or more modifications can comprise a substitution (e.g., conservative substitutions) which can comprise substitutions (e.g., at the C-terminus, at the N-terminus, or at another place in the chimeric polypeptide or segment or portion thereof) for about 1, about 2, about 3, about 4, about 5, about 6, about 7, about 8, about 9, about 10, about 11, about 12, about 13, about 14, about 15, about 16, about 17, about 18, about 19, or about 20 amino acids (e.g., natural amino acids, or modified or unusual amino acids).

A “portion” of a polypeptide is defined as at least 3 consecutive amino acids of that polypeptide. In some embodiments, a portion of a polypeptide can comprise at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 50, at least 100, at least 200, at least 500, at least 700, or at least 1000, or from about 1 to about 1000 amino acids, from about 4 to about 1000 amino acids, from about 5 to about 1000 amino acids, from about 10 to about 1000 amino acids, from about 50 to about 1000 amino acids, from about 1 to about 500 amino acids, from about 4 to about 500 amino acids, from about 5 to about 500 amino acids, from about 10 to about 500 amino acids, from about 50 to about 500 amino acids, from about 1 to about 300 amino acids, from about 4 to about 300 amino acids, from about 5 to about 300 amino acids, from about 10 to about 300 amino acids, or from about 50 to about 300 amino acids consecutive amino acids of that polypeptide.

A “functional polypeptide” is defined as a polypeptide (e.g., a wt polypeptide or a modified polypeptide) that has desired levels (e.g., at least about 20%, at least about 40%, at least about 50%, at least about 75%, at least about 80%, at least about 90%, at least about 95%, or at least about 99%, as compared to another polypeptide, such as a naturally occurring polypeptide) of one or more functions such as, for example, the ability to bind PD-1 (e.g., human PD-1, mouse PD-1, rat PD-1, monkey PD-1, mammal PD-1, primate PD-1, or SEQ ID NO:34), the ability to target T_{FH} cells, the ability to kill T_{FH} cells, or the ability to result in degranulation. In some embodiments, the functional polypeptide has an increased level of one or more functions as compared to another polypeptide (e.g., a naturally occurring polypeptide). Nucleic acid molecules can be designed to encode for functional polypeptides, and such nucleic acid molecules are encompassed by the present invention.

A “functionally equivalent” polypeptide (e.g., a chimeric polypeptide or a segment therein) is defined as a polypeptide that has been modified (e.g., by one or more insertions, one or more deletions, or one or more substitutions (e.g., conservative substitutions)) from an original polypeptide (e.g., a wt-PD-L1 portion of a chimeric polypeptide or any portion of a chimeric polypeptide) and that modified polypeptide retains desired levels (e.g., at least about 20%, at least about 40%, at least about 50%, at least about 75%, at least about 80%, at least about 90%, at least about 95%, or at least about 99%) of one or more functions of the original polypeptide, such as, for example, the ability to bind PD-1 (e.g., human PD-1, mouse PD-1, rat PD-1, monkey PD-1, mammal PD-1, primate PD-1, or SEQ ID NO:34), the ability to target T_{FH} cells, the ability to kill T_{FH} cells, or the ability to result in degranulation. In some embodiments, the functionally equivalent polypeptide has an increased level of one or more functions compared to the original polypeptide. Nucleic acid molecules can be designed to encode for functionally equivalent polypeptides, and such nucleic acid molecules are encompassed by the present invention.

In certain embodiments, the shorter the length of a polypeptide, the fewer the modifications (e.g., substitutions) that can be made within the polypeptide while retaining, for example, a desired level of a chosen function. In some instances, longer polypeptides can have a greater number of such changes while retaining, for example, a desired level of a chosen function. In other embodiments, a full-length polypeptide can have more tolerance for a fixed number of changes while retaining, for example, a desired level of a chosen function, as compared to a shorter length of that polypeptide.

The design of substitutions can take many forms, including but not limited to those described herein. In some embodiments, the hydropathic index of amino acids may be considered in designing substitutions. In the hydropathic index, each amino acid is assigned a hydropathic index on the basis of their hydrophobicity or charge characteristics, as follows: isoleucine (+4.5); valine (+4.2); Leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); or arginine (−4.5). In some instances, certain amino acids may be substituted for other amino acids having a similar hydropathic index. In making changes based upon the hydropathic index, the substitution of amino acids with hydropathic indices can be made with amino acids that have an index difference of no more than ±2, no more than ±1, or no more than ±0.5.

In some embodiments, substitutions can also be made based on hydrophilicity values. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids with hydrophilicity values can be made with amino acids that have a value of no more than ±2, no more than ±1, or no more than ±0.5.

A “conservative substitution” in an amino acid sequence, polypeptide, or a polypeptide segment indicates that a given amino acid residue is replaced by a residue having similar physiochemical characteristics (e.g., no more than ±1 when based on hydropathic index or no more than ±1 when base on hydrophilicity values). Examples of conservative substitutions include (a) substitution of one aliphatic residue for another with an aliphatic residue, (b) substitution of one of Ile, Val, Leu, or Ala for one another of Ile, Val, Leu, or Ala, (c) substitution of one of Gly, Ile, Val, Leu, or Ala for one another of Gly, Ile, Val, Leu, or Ala, (d) substitution of one polar residue for another polar residue, (e) substitution of one of Lys and Arg with another of Lys and Arg, (f) substitution of one of Glu and Asp with another of Glu and Asp, (g) substitution of one of Gln and Asn with another of Gln and Asn, (h) substitution of one hydroxyl or sulfur containing residue with another hydroxyl or sulfur containing residue, (i) substitution of one of Ser, Cys, Thr, or Met with another of Ser, Cys, Thr, or Met, (j) substitution of one aromatic residue for another with an aromatic residue, (k) substitution of one of Phe, Tyr, or Trp with another of Phe, Tyr, or Trp, (l) substitution of one basic residue for another basic residue, (m) substitution of one of His, Lys, or Arg with another of His, Lys, or Arg, (n) substitution of an acidic/amide residue with another acidic/amide residue, (o) substitution of one of Asp, Glu, Asn, or Gln with another of Asp, Glu, Asn, or Gln, (p) substitution of a residue with another residue of a similar size, and (q) substitution of one of Ala, Gly, or Ser with another of Ala, Gly, or Ser. In some embodiments, each amino acid in a hydrophobic region of a polypeptide can be substituted with conservative substitutions (e.g., any combination of conservative substitutions relating to hydrophobic residues).

While discussion has focused on amino acid changes, it will be appreciated that these changes may occur by alteration of the encoding DNA; taking into consideration also that the genetic code is degenerate and that two or more codons may code for the same amino acid. A table of amino acids and their codons is presented below for use in such embodiments, as well as for other uses, such as in the design of probes and primers and the like.

TABLES A and B
Amino acid designations and codon table

| Table A - Amino Acid Designations | | | Table B - Codons for Amino Acids |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The term “functionally equivalent codon” is used herein to refer to codons that encode the same amino acid, such as the six codons for arginine or serine.

In certain instances, the nucleic acid molecule can be engineered to contain distinct sequences while at the same time retaining the capacity to encode a desired polypeptide. In some embodiments, this can be accomplished owing to the degeneracy of the genetic code (i.e., the presence of multiple codons) which encode for the same amino acids. In other instances, it can be accomplished by including, adding, or excluding introns in the nucleic acid molecule.

In certain embodiments, a restriction enzyme recognition sequence can be introduced into a nucleic acid sequence while maintaining the ability of that nucleic acid molecule to encode a desired polypeptide. In other embodiments, a CRISPR system (e.g., a CRISPR system comprising one or more of guide RNA, crRNA, tracrRNA, sgRNA, DNA repair template, and Cas protein, such as but not limited to CRISPR/Cas9) can be used to introduce a nucleic acid molecule while maintaining the ability of that nucleic acid molecule to encode a desired polypeptide.

It will also be understood that amino acid sequences (e.g., polypeptides) and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, such as including the maintenance of biological activity where polypeptide expression is concerned. The addition of terminal sequences particularly applies to nucleic acid sequences that may, for example, include various non-coding sequences flanking either of the 5′ or 3′ portions of the coding region or may include various internal sequences, (i.e., introns) which can occur within genes.

Some embodiments of the present invention rely on or use synthesis of polypeptides in cyto, via transcription and translation of appropriate nucleic acid molecules (e.g., nucleic acid sequences as discussed herein). These polypeptides will include the twenty “natural” amino acids, and post-translational modifications thereof. In vitro peptide synthesis permits the use of modified or unusual amino acids. In some embodiments, the inventive polypeptide encompasses modifications (e.g., one or more substitutions or one or more insertions) that include one or more modified or unusual amino acids. A table of exemplary, but not limiting, modified or unusual amino acids is provided in Table C.

TABLE C
Modified or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, | AHyl | allo-Hydroxylysine |
| | beta-Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, |
| | | | sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

### Chimeric Polypeptides, Nucleic Acid Molecules, and Compositions

Some embodiments of the invention include chimeric polypeptides comprising (a) an extracellular segment, (b) a transmembrane segment, and (c) an intracellular segment. In certain embodiments, the chimeric polypeptide is a single amino acid chain. When the chimeric polypeptide is in a cell, the extracellular segment of the chimeric polypeptide is found mostly on the outside of the cell. In some embodiments, certain portions (but not all) of the extracellular segment can bind to or be within the cell membrane. In other embodiments, none of the extracellular segment binds to the cell membrane, is within the cell membrane, or both. When the chimeric polypeptide is in a cell, the transmembrane segment is found mostly within the cell membrane. In some embodiments, certain portions of the transmembrane segment can be found inside the cell, outside the cell, or both. In some embodiments, the transmembrane segment is found only within the cell membrane, or none of the transmembrane segment can be found inside the cell, outside the cell, or both. When the chimeric polypeptide is in a cell, the intracellular segment of the chimeric polypeptide is found mostly on the inside of the cell. In some embodiments, certain portions (but not all) of the intracellular segment can bind to or be within the cell membrane. In other embodiments, none of the intracellular segment binds to the cell membrane, is within the cell membrane, or both.

In certain embodiments, the chimeric polypeptide is not a naturally occurring polypeptide. In other embodiments, the chimeric polypeptide is not a wt PD-L1 (e.g., not a mammalian PD-L1, a mouse PD-L1 (e.g., UniProt No. E2D891 or UniProt No. Q9EP73), a dog PD-L1 (e.g., GenBank: BAO74172.1), a cat PD-L1 (e.g., UniProt No. A8WEV0), a rat PD-L1, a pig PD-L1 (e.g., UniProt Q4QTK1), a woodchuck PD-L1 (e.g., UniProt B8PXK6), a cow PD-L1 (e.g., GenBank: BAH82662.1), a monkey PD-L1 (e.g., a cynomolgus monkey), a primate PD-L1, or a human PD-L1 UnitProt Q9NZQ7-1, UnitProt Q9NZQ7-2, or UnitProt Q9NZQ7-3). In some embodiments, the chimeric polypeptide does not comprise an Fc region of an antibody (e.g., a wt antibody). In some embodiments, one or more of the extracellular segments, intracellular segments, or transmembrane segments do not comprise an Fc region of an antibody (e.g., a wt antibody).

In other embodiments of the chimeric polypeptide, the extracellular segment and the transmembrane segment are not both present in the same endogenous single-chain polypeptide in a mammal. In still other embodiments, the extracellular segment and the intracellular segment are not both present in the same endogenous single-chain polypeptide in a mammal. In yet other embodiments, the intracellular segment and the transmembrane segment are not both present in the same endogenous single-chain polypeptide in a mammal. In certain embodiments, the extracellular segment, the transmembrane segment, and the intracellular segment are not all present in the same endogenous single-chain polypeptide in a mammal.

In some embodiments, the chimeric polypeptide (e.g., when in a cell, an immune cell, an NK cell, or a T cell) (a) targets follicular helper T cells (T_{FH}), (b) kills follicular helper T cells (T_{FH}), (c) has an extracellular segment that binds to programmed cell death protein 1 (PD-1, such as, for example, human PD-1, mouse PD-1, rat PD-1, monkey PD-1, mammal PD-1, primate PD-1, or SEQ ID NO:34), (d) results in degranulation, (e) activates the cell (e.g., an immune cell) or (f) combinations thereof. In some embodiments, the chimeric polypeptide comprises one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO:14, SEQ ID NO: 15, SEQ ID NO: 16, SEQ ID NO:17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:38, combinations thereof, or one or more modifications thereof. In some embodiments, the chimeric polypeptide comprises SEQ ID NO:25, SEQ ID NO:26, SEQ ID NO:27, SEQ ID NO:28, SEQ ID NO:29, SEQ ID NO:30, SEQ ID NO:31, or one or more modifications thereof.

In certain embodiments, the extracellular segment is linked to the transmembrane segment by a linker with one or more amino acids (e.g., about 1, about 2, about 3, about 4, about 5, about 6, about 7, about 8, about 9, about 10, about 15, about 20, about 25, about 50, about 100, about 200, or about 500 amino acids, or from about 1 to about 50, from about 1 to about 200, or from about 1 to about 500). In other embodiments, the transmembrane segment is linked to the intracellular segment by a linker with one or more amino acids (e.g., about 1, about 2, about 3, about 4, about 5, about 6, about 7, about 8, about 9, about 10, about 15, about 20, about 25, about 50, about 100, about 200, or about 500 amino acids, or from about 1 to about 50, from about 1 to about 200, or from about 1 to about 500).

The extracellular segment comprises wild type programmed cell death ligand 1 (wt-PD-L1), wt-PD-L1 with one or more modifications, or a portion of wt-PD-L1 with optionally one or more modifications. PD-L1 is also known as CD274 and B7-H1. The wt-PD-L1 includes any full length naturally occurring PD-L1 (i.e., wt PD-L1), such as, but not limited to, a mammalian PD-L1, a mouse PD-L1 (e.g., UniProt No. E2D891 or UniProt No. Q9EP73), a dog PD-L1 (e.g., GenBank: BAO74172.1), a cat PD-L1 (e.g., UniProt No. A8WEV0), a rat PD-L1, a pig PD-L1 (e.g., UniProt Q4QTK1), a woodchuck PD-L1 (e.g., UniProt B8PXK6), a cow PD-L1 (e.g., GenBank: BAH82662.1), a monkey PD-L1 (e.g., a cynomolgus monkey), a primate PD-L1, or a human PD-L1 (e.g., SEQ ID NO:38 (UnitProt Q9NZQ7-1), UnitProt Q9NZQ7-2, or UnitProt Q9NZQ7-3).

In some embodiments, the extracellular segment comprises the amino acid sequence ADYKR (SEQ ID NO:14) or ADYK (SEQ ID NO:15). In some embodiments, the extracellular segment comprises about 3, about 4, about 5, about 10, about 15, about 20, about 25, about 30, about 35, about 50, about 100, about 150, about 200, about 250, about 350, about 400, about 450, about 500, about 750, or about 1000 amino acids, or about 2000 amino acids, or from about 3 to about 2000 amino acids, from about 4 to about 2000 amino acids, from about 5 to about 2000 amino acids, from about 10 to about 2000 amino acids, from about 50 to about 2000 amino acids, from about 3 to about 1000 amino acids, from about 4 to about 1000 amino acids, from about 5 to about 1000 amino acids, from about 10 to about 1000 amino acids, from about 50 to about 1000 amino acids, from about 3 to about 500 amino acids, from about 4 to about 500 amino acids, from about 5 to about 500 amino acids, from about 10 to about 500 amino acids, from about 50 to about 500 amino acids, from about 3 to about 300 amino acids, from about 4 to about 300 amino acids, from about 5 to about 300 amino acids, from about 10 to about 300 amino acids, or from about 50 to about 300 amino acids. In other embodiments, the Kd (dissociation constant) of the extracellular segment (or of the chimeric polypeptide) with PD-1 (e.g., human PD-1, mouse PD-1, rat PD-1, monkey PD-1, mammal PD-1, primate PD-1, or SEQ ID NO:34) is about 100 nm, about 200 nm, about 300 nm, about 400 nm, about 500 nm, about 750 nm, about 1000 nm, about 2000 nm, about 3000 nm, about 4000 nm, about 5000 nm, about 7000 nm, about 8000 nm, about 9000 nm, about 10000 nm, about 15000 nm, or about 20000 nm, or from about 100 nm to about 20000 nm, from about 100 nm to about 10000 nm, from about 500 nm to about 20000 nm, from about 500 nm to about 10000 nm, from about 500 nm to about 9000 nm, from about 750 nm to about 10000 nm, or from about 750 nm to about 9000 nm.

With regard to the extracellular segment, one or more modifications to PD-L1, in some instances, can include an insertion, a deletion, a substitution, or combinations thereof. In certain embodiments, one or more modifications to a wt-PD-L1 can comprise an insertion which can comprise inserting (e.g., at the C-terminus, at the N-terminus, or at another place in the polypeptide or segment or portion thereof) about 1, about 2, about 3, about 4, about 5, about 6, about 7, about 8, about 9, about 10, about 11, about 12, about 13, about 14, about 15, about 16, about 17, about 18, about 19, or about 20 amino acids (e.g., natural amino acids, or modified or unusual amino acids). In certain embodiments, one or more modifications to a wt-PD-L1 can comprise a deletion which can comprise deleting (e.g., at the C-terminus, at the N-terminus, or at another place in the chimeric polypeptide or segment or portion thereof) about 1, about 2, about 3, about 4, about 5, about 6, about 7, about 8, about 9, about 10, about 11, about 12, about 13, about 14, about 15, about 16, about 17, about 18, about 19, or about 20 amino acids. In certain embodiments, one or more modifications to a wt-PD-L1 can comprise a substitution (e.g., conservative substitutions) which can comprise substitutions (e.g., at the C-terminus, at the N-terminus, or at another place in the chimeric polypeptide or segment or portion thereof) for about 1, about 2, about 3, about 4, about 5, about 6, about 7, about 8, about 9, about 10, about 11, about 12, about 13, about 14, about 15, about 16, about 17, about 18, about 19, or about 20 amino acids (e.g., natural amino acids, or modified or unusual amino acids). In certain embodiments, the modified PD-L1 results in a chimeric polypeptide that is a functional PD-L1 polypeptide (e.g., the ability to bind PD-1 (e.g., human PD-1, mouse PD-1, rat PD-1, monkey PD-1, mammal PD-1, primate PD-1, or SEQ ID NO:34—see also ZAK et al. (2017) “Structural Biology of the Immune Checkpoint Receptor PD-1 and Its Ligands PD-L1/PD-L2” Structure, Vol. 25, pp. 1163-1174 (with supplemental information), which is incorporated herein by reference in its entirety), the ability to target T_{FH} cells, the ability to kill T_{FH} cells, or the ability to result in degranulation). In other embodiments, the modified PD-L1 results in a chimeric polypeptide that is functionally equivalent to a wt-PD-L1 polypeptide with regard to, for example, the ability to bind PD-1 (e.g., human PD-1, mouse PD-1, rat PD-1, monkey PD-1, mammal PD-1, primate PD-1, or SEQ ID NO:34), the ability to target T_{FH} cells, the ability to kill T_{FH} cells, or the ability to result in degranulation.

In some embodiments, the extracellular segment does not encompass one or more naturally occurring polypeptides (e.g., does not encompass one or more of the wt-PD-L1s, e.g., human PD-1, mouse PD-1, rat PD-1, monkey PD-1, mammal PD-1, primate PD-1, or SEQ ID NO:34). In other embodiments, the extracellular segment does not encompass any of the wt-PD-L1s (e.g., human PD-1, mouse PD-1, rat PD-1, monkey PD-1, mammal PD-1, primate PD-1, or SEQ ID NO:34). In some embodiments, the extracellular segment does not encompass any naturally occurring polypeptide (e.g., does not encompass any of the wt-PD-L1 or any other naturally occurring polypeptide).

In some embodiments, one or more modifications to a wt-PD-L1 can include one or more substitutions, one or more insertions, or one or more deletions (or combinations thereof) to one or more amino acids in a hydrophobic region of a wt-PD-L1, in a signal region of a wt-PD-L1, in a transmembrane region of a wt-PD-L1, or in a combination thereof. In some embodiments, one or more modifications to a wt-PD-L1 can include one or more substitutions or one or more deletions (or combinations thereof) to one or more amino acids in a hydrophobic region of a wt-PD-L1, in a signal region of a wt-PD-L1, in a transmembrane region of a wt-PD-L1, or in a combination thereof.

In some embodiments, the extracellular segment can comprise a polypeptide sequence with an amino acid sequence identity to a wt-PD-L1 (e.g., a mammalian PD-L1, a mouse PD-L1 (e.g., UniProt No. E2D891 or UniProt No. Q9EP73), a dog PD-L1 (e.g., GenBank: BAO74172.1), a cat PD-L1 (e.g., UniProt No. A8WEV0), a rat PD-L1, a pig PD-L1 (e.g., UniProt Q4QTK1), a woodchuck PD-L1 (e.g., UniProt B8PXK6), a cow PD-L1 (e.g., GenBank: BAH82662.1), a monkey PD-L1 (e.g., a cynomolgus monkey), a primate PD-L1, or a human PD-L1 (e.g., SEQ ID NO:38 (UnitProt Q9NZQ7-1), UnitProt Q9NZQ7-2, or UnitProt Q9NZQ7-3)) of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. In some embodiments, the extracellular segment sequence has an amino acid sequence identity to a mammalian PD-L1, a mouse PD-L1 (e.g., UniProt No. E2D891 or UniProt No. Q9EP73), a dog PD-L1 (e.g., GenBank: BAO74172.1), a cat PD-L1 (e.g., UniProt No. A8WEV0), a rat PD-L1, a pig PD-L1 (e.g., UniProt Q4QTK1), a woodchuck PD-L1 (e.g., UniProt B8PXK6), a cow PD-L1 (e.g., GenBank: BAH82662.1), a monkey PD-L1 (e.g., a cynomolgus monkey), a primate PD-L1, or a human PD-L1 (e.g., SEQ ID NO:38 (UnitProt Q9NZQ7-1), UnitProt Q9NZQ7-2, or UnitProt Q9NZQ7-3) of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. The amino acid sequence identity (e.g., percent identity) can be determined by any suitable method, such as using BLAST, BLAST-2, ALIGN, ALIGN-2, Clustal Omega, or Megalign software. Unless otherwise indicated, the amino acid sequence identity (e.g., percent identity) is determined using BLAST-2.

In some embodiments, the extracellular segment optionally comprises one or more (e.g., 1, 2, 3, 4, or 5) spacer sequences. In certain embodiments, one or more of the spacer sequences (a) are between the PD-L1 portion (e.g., wt-PD-L1 or a modification thereof) of the extracellular segment and the transmembrane segment, (b) are not between the wt-PD-L1 portion (e.g., PD-L1 or a modification thereof) of the extracellular segment and the transmembrane segment, or (c) both. In some embodiments, each of the one or more spacer sequences can comprise up to about 1000 amino acids (e.g., about 1, about 5, about 10, about 20, about 25, about 50, about 100, about 150, about 200, about 250, about 300 amino acids, about 500 amino acids, or about 1000 amino acids, from 5 to about 25, from 5 to about 50, from 5 to about 100, from 5 to about 300, from 5 to about 500, from 5 to about 1000 amino acids, or 1 to about 1000 amino acids). In other embodiments, one or more spacer sequences can be a polypeptide (e.g., from an animal, mammal, mouse, rat, primate, monkey, or human) comprising variable regions of an antibody (e.g. H chain and L chain), Fc regions of an antibody, TCR (T cell receptor) (e.g., TCRα, TCRβ, TCRγ, or TCRδ), CD4 ectodomain, CD8α, CD8β, CD11A, CD11B, CD11C, CD18, CD29, CD49A, CD49B, CD49D, CD49E, CD49F, CD61, CD41, or CD51, or one or more modifications thereof, or combinations thereof. In some embodiments, one or more spacer sequences can be a polypeptide (e.g., from an animal, mammal, mouse, rat, primate, monkey, or human) comprising or consisting of CD8α, a portion of CD8a (e.g., a hinge sequence or a leader sequence), CD8β, a portion of CD8β, CD4, a portion of CD4, CD28, or a portion of CD28, or one or more modifications thereof, or combinations thereof. In some embodiments, the spacer sequence can be a polypeptide comprising SEQ ID NO.: 13, amino acid numbers 118 to 178 of CD8a (NCBI Ref Seq: NP_001759.3) which is a hinge sequence of CD8α, CD8a leader sequence (e.g., SEQ ID NO:10), amino acid numbers 135 to 195 of CD8β (GenBank: AAA35664.1), amino acid numbers 315 to 396 of CD4 (NCBI Ref Seq: NP_000607.1), amino acid numbers 114 to 152 of CD28 (NCBI Ref Seq: NP_006130.1), or a portion thereof, or one or more modifications thereof. In other embodiments, the one or more spacer sequences may be an artificially synthesized sequence.

In some embodiments, the extracellular segment comprises one or more of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, or a portion thereof, or one or more modifications thereof.

In some embodiments, the transmembrane segment can comprise a wt polypeptide, a modification of a wt polypeptide, or an artificially designed polypeptide. For example, the transmembrane segment can be a polypeptide portion (or modification thereof) obtained from any membrane-binding or transmembrane protein or polypeptide. In certain embodiments, the transmembrane segment can comprise of a T cell receptor α or β chain, a CD3ζ chain, CD28, CD3ε, CD45, CD4, CD5, CD8, CD9, CD16, CD22, CD33, CD37, CD64, CD80, CD86, CD134, CD137, Inducible T-cell COStimulator (ICOS), CD154, or a glucocorticoid-induced TNFR-related protein (GITR), or portions, or one or more modifications thereof. In other embodiments, the artificially designed transmembrane segment can be a polypeptide comprising hydrophobic residues such as leucine and valine. In some embodiments, a triplet of phenylalanine, tryptophan and valine can be found at each end of the synthetic transmembrane segment. In some embodiments, the transmembrane segment comprises about 3, about 4, about 5, about 10, about 15, about 20, about 25, about 30, about 35, about 50, about 100, about 150, about 200, about 250, about 350, about 400, about 450, about 500, about 750, or about 1000 amino acids, or from about 3 to about 1000 amino acids, from about 4 to about 1000 amino acids, from about 5 to about 1000 amino acids, from about 10 to about 1000 amino acids, from about 50 to about 1000 amino acids, from about 3 to about 500 amino acids, from about 4 to about 500 amino acids, from about 5 to about 500 amino acids, from about 10 to about 500 amino acids, from about 50 to about 500 amino acids, from about 3 to about 300 amino acids, from about 4 to about 300 amino acids, from about 5 to about 300 amino acids, from about 10 to about 300 amino acids, or from about 50 to about 300 amino acids. In certain embodiments, a linker (e.g., a linker having a length of about 1 to about 10 amino acids, of about 1 to about 25 amino acids, of about 1 to about 50 amino acids, or of about 1 to about 100 amino acids) can be between the transmembrane segment and the intracellular segment or between the transmembrane segment and the extracellular segment, as described herein (e.g., a linker sequence having a glycine-serine continuous sequence), or both. In certain embodiments, the transmembrane segment can be SEQ ID NO:16, SEQ ID NO:17, the sequence of amino acid numbers 153 to 179 of CD28 (NCBI Ref Seq: NP_006130.1), or portions thereof, or one or more modifications thereof.

In some embodiments, the intracellular segment can comprise one or more polypeptides to transmit a signal into a cell (e.g., immune cell, NK cells or T Cells) when the extracellular segment of the chimeric polypeptide interacts with (e.g., binds to) another molecule (e.g., on another cell). In certain embodiments, a signal can be any suitable signal, including but not limited to phosphorylation or de-phosphorylation, a signal that results in phosphorylation or de-phosphorylation, association or dissociation of intracellular molecules, a signal that results in association or dissociation of intracellular molecules, a signal that results in increasing or decreasing intracellular ion concentrations (e.g., Ca), a signal that results in increasing or decreasing gene expression, a signal that results in degranulation, or any other signaling activity found in immune cells (e.g., NK cells or T Cells). In some embodiments, the intracellular portions (or modifications thereof) from other polypeptides (e.g., signal transmitting polypeptides) can be used. Any suitable signal transmitting polypeptide can be used including but not limited to cytoplasmic sequences from a TCR complex or a costimulatory molecule, or portions thereof, or one or more modifications thereof (e.g., to be functionally equivalent).

In some embodiments, the intracellular segment comprises about 3, about 4, about 5, about 10, about 15, about 20, about 25, about 30, about 35, about 50, about 100, about 150, about 200, about 250, about 350, about 400, about 450, about 500, about 750, or about 1000 amino acids, or about 2000 amino acids, or from about 3 to about 2000 amino acids, from about 4 to about 2000 amino acids, from about 5 to about 2000 amino acids, from about 10 to about 2000 amino acids, from about 50 to about 2000 amino acids, from about 3 to about 1000 amino acids, from about 4 to about 1000 amino acids, from about 5 to about 1000 amino acids, from about 10 to about 1000 amino acids, from about 50 to about 1000 amino acids, from about 3 to about 500 amino acids, from about 4 to about 500 amino acids, from about 5 to about 500 amino acids, from about 10 to about 500 amino acids, from about 50 to about 500 amino acids, from about 3 to about 300 amino acids, from about 4 to about 300 amino acids, from about 5 to about 300 amino acids, from about 10 to about 300 amino acids, or from about 50 to about 300 amino acids.

In some embodiments, the intracellular segment of the chimeric polypeptide can comprise a first signaling polypeptide and optionally one or more second signaling polypeptides. In some embodiments, the signaling of the first signaling polypeptide can be dependent on the extracellular segment interacting (e.g., binding) with another molecule. In other embodiments, the signaling of the one or more second signaling polypeptides is not directly dependent on the extracellular segment interacting (e.g., binding) with another molecule.

In some embodiments, the first signaling polypeptide comprises about 3, about 4, about 5, about 10, about 15, about 20, about 25, about 30, about 35, about 50, about 100, about 150, about 200, about 250, about 350, about 400, about 450, about 500, about 750, or about 1000 amino acids, or about 2000 amino acids, or from about 3 to about 2000 amino acids, from about 4 to about 2000 amino acids, from about 5 to about 2000 amino acids, from about 10 to about 2000 amino acids, from about 50 to about 2000 amino acids, from about 3 to about 1000 amino acids, from about 4 to about 1000 amino acids, from about 5 to about 1000 amino acids, from about 10 to about 1000 amino acids, from about 50 to about 1000 amino acids, from about 3 to about 500 amino acids, from about 4 to about 500 amino acids, from about 5 to about 500 amino acids, from about 10 to about 500 amino acids, from about 50 to about 500 amino acids, from about 3 to about 300 amino acids, from about 4 to about 300 amino acids, from about 5 to about 300 amino acids, from about 10 to about 300 amino acids, or from about 50 to about 300 amino acids. In some embodiments, one of the optional second signaling polypeptide comprises about 3, about 4, about 5, about 10, about 15, about 20, about 25, about 30, about 35, about 50, about 100, about 150, about 200, about 250, about 350, about 400, about 450, about 500, about 750, or about 1000 amino acids, or about 1500 amino acids, or from about 3 to about 1500 amino acids, from about 4 to about 2000 amino acids, from about 5 to about 1500 amino acids, from about 10 to about 1500 amino acids, from about 50 to about 1500 amino acids, from about 3 to about 1000 amino acids, from about 4 to about 1000 amino acids, from about 5 to about 1000 amino acids, from about 10 to about 1000 amino acids, from about 50 to about 1000 amino acids, from about 3 to about 500 amino acids, from about 4 to about 500 amino acids, from about 5 to about 500 amino acids, from about 10 to about 500 amino acids, from about 50 to about 500 amino acids, from about 3 to about 300 amino acids, from about 4 to about 300 amino acids, from about 5 to about 300 amino acids, from about 10 to about 300 amino acids, or from about 50 to about 300 amino acids.

In certain embodiments, the first signaling polypeptide can be any suitable polypeptide that can stimulate (directly or indirectly) the activation of an immune cell (e.g., an NK cell or a T cell). In other embodiments, the first signaling polypeptide can comprise a signal transduction motif known as an immunoreceptor tyrosine-based activation motif (ITAM) (e.g., two Tyr-x-x-Leu/Ile elements (where x is any amino acid) separated by six to eight amino acid residues) (e.g., see, RETH (1989) “Antigen receptor tail clue” Nature, Vol. 338, pp. 383-384, which is incorporated herein by reference in its entirety). In other embodiments, the first signaling polypeptide can comprise a signal transduction motif known as an immunoreceptor tyrosine-based inhibition motif (ITIM) (e.g., Ile/Val/Leu/Ser-x-Tyr-x-x-Leu/Val, where x denotes any amino acid) (e.g., see BURSHTYN et al. (1999) “Conserved Residues Amino-Terminal of Cytoplasmic Tyrosines Contribute to the SHP-1-Mediated Inhibitory Function of Killer Cell Ig-Like Receptors” J Immunol., Vol. 162, No. 2, pp. 897-902, which is incorporated herein by reference in its entirety).

In some embodiments, the first signaling polypeptide can comprise CD3ζ, FcRγ, FcRβ, CD3γ, CD3δ, CD3ε, CD5, CD22, CD79a, CD79b, or CD66d, or portions thereof, or one or more modifications thereof. In certain embodiments, the first signaling polypeptide can comprise SEQ ID NO: 18, SEQ ID NO: 19, SEQ ID NO: 20, amino acid numbers 51 to 164 of CD3ζ (NCBI Ref Seq: NP 932170.1), amino acid numbers 45 to 86 of FcεRIγ (NCBI Ref Seq: NP 004097.1), amino acid numbers 201 to 244 of FcεRIβ (NCBI Ref Seq: NP 000130.1), amino acid numbers 139 to 182 of CD3γ (NCBI Ref Seq: NP_000064.1), amino acid numbers 128 to 171 of CD3δ (NCBI Ref Seq: NP_000723.1), amino acid numbers 153 to 207 of CD3ε (NCBI Ref Seq: NP 000724.1), amino acid numbers 402 to 495 of CD5 (NCBI Ref Seq: NP 055022.2), amino acid numbers 707 to 847 of CD22 (NCBI Ref Seq: NP_001762.2), amino acid numbers 166 to 226 of CD79a (NCBI Ref Seq: NP_001774.1), amino acid numbers 182 to 229 of CD79b (NCBI Ref Seq: NP 000617.1), or amino acid numbers 177 to 252 of CD66d (NCBI Ref Seq: NP 001806.2), or one or more modifications thereof.

In some embodiments, the second signaling polypeptide can comprise CD2, CD4, CD5, CD8α, CD8β, CD28, CD134, CD137, ICOS, or CD154, or portions thereof, or one or more modifications thereof. In some embodiments, the second signaling polypeptide can comprise SEQ ID NO: 21, SEQ ID NO: 22, amino acid numbers 236 to 351 of CD2 (NCBI Ref Seq: NP 001758.2), amino acid numbers 421 to 458 of CD4 (NCBI Ref Seq: NP 000607.1), amino acid numbers 402 to 495 of CD5 (NCBI Ref Seq: NP 055022.2), amino acid numbers 207 to 235 of CD8a (NCBI Ref Seq: NP 001759.3), amino acid numbers 196 to 210 of CD8β (GenBank: AAA35664.1), amino acid numbers 181 to 220 of CD28 (NCBI Ref Seq: NP 006130.1), amino acid numbers 214 to 255 of CD137 (4-1BB, NCBI Ref Seq: NP_001552.2), amino acid numbers 241 to 277 of CD134 (OX40, NCBI Ref Seq: NP 003318.1), or amino acid numbers 166 to 199 of ICOS (NCBI Ref Seq: NP 036224.1), or one or more modifications thereof.

In some embodiments, linkers (which can be the same or different) can be between the first signaling polypeptide and a second signaling polypeptide or between two second signaling polypeptides. The linkers can be, for example, 1, 2, 3, 4, 5, 6, 7, 8, 9, 10, 15, 20, 30, 40, 50, or 100 amino acids (e.g., from 2 to 10 amino acids). In some instances, a linker having a glycine-serine continuous sequence can be used.

In some embodiments, the intracellular segment comprises SEQ ID NO: 18, SEQ ID NO: 19, SEQ ID NO: 20, SEQ ID NO: 21, SEQ ID NO: 22, SEQ ID NO: 23, SEQ ID NO: 24, or one or more modifications thereof.

In some embodiments, the chimeric polypeptide comprises SEQ ID NO: 25, SEQ ID NO: 26, SEQ ID NO: 27, SEQ ID NO: 28, SEQ ID NO: 29, SEQ ID NO: 30, SEQ ID NO: 31, or one or more modifications thereof.

Some embodiments of the invention include nucleic acid molecules that can encode for the chimeric polypeptide (“chimeric nucleic acid molecules”). In certain embodiments, the chimeric nucleic acid molecule is included in a vector (e.g., a viral vector, a retroviral vector, a lentiviral vector, an adenoviral vector, an adeno-associated viral vector, a herpesviral vector, a chimeric viral vector, a plasmid, a cosmid, an artificial chromosome, a bacteriophage, an animal virus, a plant virus, an expression vector, a conjugative vector, a nonconjugative vector or an mRNA transfection). In certain embodiments, the chimeric nucleic acid molecule is in a cell, such as an insect cell (e.g., an Sf9 cell) or a mammalian cell (e.g., a human cell, a rat cell, a mouse cell, a CHO cell, an immune cell, an immune cell progenitor, a T cell, a T cell progenitor, an NK cell, KHYG1, NK-92, YT, SNK-6, NKL, or an NK cell progenitor). In certain embodiments, the chimeric nucleic acid molecule is in a cell and the cell is isolated from an animal, a mammal, a primate, or a human.

In other embodiments, the chimeric nucleic acid molecule comprises one or more nucleic acid sequences that are not used to encode for the chimeric polypeptide (e.g., one or more introns). For example, the chimeric nucleic acid molecule can comprise a nucleic acid sequence as found in nature (e.g., including introns). In certain embodiments, the chimeric nucleic acid molecule differs from the one or more nucleic acid molecules in nature because the chimeric nucleic acid molecule does not include one or more introns. In some embodiments, the chimeric nucleic acid molecule is a cDNA molecule (“chimeric cDNA molecule”). In certain embodiments, the segments of the chimeric cDNA molecule are identical to a nucleic acid molecule found in nature. In other embodiments, the segments of the chimeric cDNA molecule are not identical to a nucleic acid molecule found in nature (e.g., due to a segment of the cDNA molecule not including one or more introns in the nucleic acid molecule found in nature).

In some embodiments, the chimeric nucleic acid molecule sequence has a sequence identity to a nucleic acid molecule encoding a chimeric polypeptide (e.g., SEQ ID NO:32) of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 90%, at least about 95%, at least about 99%, or at least about 99.5%. In some embodiments, the chimeric nucleic acid molecule sequence has a sequence identity to SEQ ID NO:32 of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 90%, at least about 95%, at least about 99%, or at least about 99.5%. The nucleic acid sequence identity (e.g., percent identity) can be determined by any suitable method, such as using BLAST, BLAST-2, ALIGN, ALIGN-2, Clustal Omega, or Megalign software. Unless otherwise indicated, the nucleic acid sequence identity (e.g., percent identity) is determined using BLAST-2.

The chimeric nucleic acid molecule can be made using any suitable technique, such as but not limited to, chemical synthesis, enzymatic production or biological production. Chemical synthesis of a nucleic acid molecule can include, for example, a nucleic acid molecule made by in vitro chemical synthesis using phosphotriester, phosphite or phosphoramidite chemistry and solid phase techniques, or via deoxynucleoside H-phosphonate intermediates. Enzymatically produced nucleic acid molecules can be accomplished using any suitable method including but not limited to Polymerase Chain Reaction (PCR). Biologically produced nucleic acid molecules can be accomplished using any suitable method including but not limited to a recombinant nucleic acid produced (i.e., replicated) in a living cell, such as a recombinant DNA vector replicated in bacteria.

Modifications or changes made in the structure of the nucleic acid molecules and/or polypeptides of the present invention are encompassed within some embodiments of the present invention. In certain embodiments, a polypeptide can be modified (e.g., by one or more insertions, one or more deletions, or one or more substitutions (e.g., conservative substitutions)). In some embodiments, the polypeptide which was modified does not have an appreciable loss (e.g., a decrease in a function of less than about 1%, less than about 5%, less than about 10%, less than about 25%, less than about 50%, less than about 75%, less than about 90%, less than about 95%, less than about 99%, or less than about 100%) of one or more chosen functions of the unmodified polypeptide such as, for example, targeting T_{FH}, killing T_{FH}, binding to PD-1 (e.g., human PD-1, mouse PD-1, rat PD-1, monkey PD-1, mammal PD-1, primate PD-1, or SEQ ID NO:34), or inducing degranulation. In some embodiments, the polypeptide which was modified retains desired levels (e.g., at least about 20%, at least about 40%, at least about 50%, at least about 75%, at least about 80%, at least about 90%, at least about 95%, or at least about 99%) of one or more functions of the unmodified polypeptide, such as, for example, targeting T_{FH}, killing T_{FH}, binding to PD-1 (e.g., human PD-1, mouse PD-1, rat PD-1, monkey PD-1, mammal PD-1, primate PD-1, or SEQ ID NO:34), or inducing degranulation. In some embodiments, the polypeptide after modification has an increased level of one or more functions as compared to the unmodified polypeptide. Nucleic acid molecules can be designed to encode for such a modified polypeptide, and such nucleic acid molecules are encompassed by the present invention.

The presently disclosed subject matter further includes a method of producing a chimeric polypeptide. Any suitable method can used to make the chimeric polypeptides including but not limited to expression through any suitable molecular biological technique (e.g., using a prokaryotic or eukaryotic expression system), isolation from a source in nature, or chemical synthesis. Eukaryotic expression systems include plant-based systems; insect cell systems via recombinant baculoviruses; whole insect systems via recombinant baculoviruses; genetically engineered yeast systems, including but not limited to Saccharomyces sp. and Pichia spp.; and mammalian cell systems, including but not limited to C2C12 cells, 10T ½ fibroblasts, NIH/3T3 fibroblasts, mesenchymal stem cells (MSCs), hematopoietic stem cells, Chinese hamster ovary cells, immune cells, NK cells, T cells, or cell lines commonly used for industrial scale expression of recombinant proteins. In some embodiments, useful plant-based expression systems can include transgenic plant systems. In some embodiments, useful plant-based expression systems can include transplastomic plant systems.

In some embodiments, a method of producing the chimeric polypeptide includes providing a host cell comprising a nucleic acid molecule, as disclosed herein, operatively linked to a promoter operable under conditions whereby the encoded polypeptide is expressed; and recovering the polypeptide from the host cell.

### Cells Comprising a Chimeric Polypeptide or a Chimeric Nucleic Acid Molecule

Some embodiments of the invention include cells comprising a chimeric polypeptide, a chimeric nucleic acid molecule or both.

Adding (e.g., transfection, transformation, or transduction) a chimeric nucleic acid molecule to a cell can be accomplished using any suitable method including but not limited to one or more of transformation (as used herein transfection methods are encompassed by the term transformation), viral transformation (e.g., using a viral vector, a retroviral vector, a lentiviral vector, an adenoviral vector, an adeno-associated viral vector, a herpesviral vector, a chimeric viral vector, a plasmid, a cosmid, an artificial chromosome, a bacteriophage, a virus, an animal virus, a plant virus, an expression vector, a conjugative vector, a nonconjugative vector or an mRNA transfection), injection, microinjection, electroporation, sonication, calcium ion treatment, calcium phosphate precipitation, PEG-DMSO treatment, DE-Dextran treatment, liposome mediated transformation, or a receptor mediated transformation. Adding a chimeric polypeptide to modify a cell can be accomplished using any suitable method including but not limited to one or more of injection, microinjection, electroporation, sonication, calcium ion treatment, calcium phosphate precipitation, PEG-DMSO treatment, DE-Dextran treatment, or liposome mediated. The added nucleic acid molecule can be part of a vector (e.g., a viral vector, a retroviral vector, a lentiviral vector, an adenoviral vector, an adeno-associated viral vector, a herpesviral vector, a chimeric viral vector, a plasmid, a cosmid, an artificial chromosome, a bacteriophage, an animal virus, a plant virus, an expression vector, a conjugative vector, a nonconjugative vector, or an mRNA transfection), a plasmid, a cosmid, an artificial chromosome, a bacteriophage, a virus, an animal virus, or a plant virus. In some embodiments, the added nucleic acid molecule is exogenous; “exogenous” means (a) that the added nucleic acid molecule originates from outside of the cell (e.g., is foreign to the cell). In some embodiments, the added polypeptide is exogenous; “exogenous” in this context means that the added polypeptide originates from outside of the cell (e.g., is foreign to the cell).

The cell can be any suitable cell including but not limited to an insect cell (e.g., an Sf9 cell), a vertebrate cell, or a mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, an adipose stem cell, an immune cell, a T cell, or an NK cell (e.g., KHYG1, NK-92, YT, SNK-6, or NKL cells)). In certain embodiments, an cell can be any suitable cell including but not limited insect cell, a vertebrate cell, or a mammalian cell (e.g., a human cell, a rat cell a mouse cell, a NIH/3T3 cell, a CHO cell, a mesenchymal stem cell (MSC), a hematopoietic stem cell, a blood cell, a bone marrow cell, a stem cell, an adipose stem cell, an immune cell, a T cell, or an NK cell (e.g., KHYG1, NK-92, YT, SNK-6, or NKL cells)). In other embodiments, the cell is autologous, allogeneic, a peripheral blood NK cell, a cord blood (allogeneic) NK cell, induced pluripotent stem cells, or an iPSC-derived NK cells (autologous or allogeneic).

In some embodiment, a process for producing a cell comprising a chimeric polypeptide, a chimeric nucleic acid molecule or both (e.g., a cell expressing the chimeric polypeptide) can comprise a step of introducing a chimeric nucleic acid molecule into a cell using any method disclosed herein. In certain embodiments, the step can be carried out ex vivo. For example, a cell can be transformed ex vivo with a virus vector or a non-virus vector carrying a chimeric nucleic acid molecule to produce a cell expressing a chimeric polypeptide. In certain embodiments, the cell can be a cell from a mammal, a primate, a monkey, a human, a mouse, a rat, a pig, a horse, or a dog.

The cell used in the process of the present disclosure is not particularly limited, and any suitable cell can be used. In certain embodiments, a cell can be collected, isolated, and/or purified from a body fluid, a tissue or an organ such as blood (e.g., peripheral blood or umbilical cord blood) or bone marrow or a cell obtained by differentiating or reprogramming the aforementioned cell to produce an induced pluripotent stem cell (iPSC) can be used. Any suitable cell can used including but not limited to a peripheral blood mononuclear cell (PBMC), an immune cell (e.g., a T cell, a dendritic cell, a B cell, a hematopoietic stem cell, a macrophage, a monocyte, a NK cell (e.g., KHYG1, NK-92, YT, SNK-6, or NKL cells) or a hematopoietic cell (e.g., a neutrophil or a basophil), an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In certain embodiments, an NK cell (e.g., KHYG1, NK-92, YT, SNK-6, or NKL cells), a T cell, a precursor cell of a T cell (e.g., a hematopoietic stem cell or a lymphocyte precursor cell) or a cell population containing them can be used. Any suitable T cell can be used including but not limited a CD8-positive T cell, a CD4-positive T cell, a regulatory T cell, a cytotoxic T cell, and a tumor infiltrating lymphocyte. Any suitable NK cell can be used including but not limited to KHYG1, NK-92, YT, SNK-6, or NKL cells. In certain embodiments, the aforementioned cells may be collected from an animal (e.g., mammal, primate, or human), obtained by expansion culture of a cell collected from an animal (e.g., mammal, primate, or human), established as a cell strain, or a combination thereof. For example, when transplantation of the produced chimeric polypeptide-expressing cell or a cell differentiated from the produced chimeric polypeptide-expressing cell into an animal (e.g., mammal, primate, or human) may in some instances be desired, the chimeric nucleic acid molecule can be introduced into a cell collected from the an animal (e.g., mammal, primate, or human) itself or a conspecific an animal (e.g., mammal, primate, or human) thereof. In certain embodiments, the chimeric nucleic acid molecule can be inserted into a vector (e.g., as disclosed herein), and the vector can be introduced into a cell. For example, a virus vector such as a retrovirus vector (including an oncoretrovirus vector, a lentivirus vector, and a pseudotyped vector), an adenovirus vector, an adeno-associated virus (AAV) vector, a simian virus vector, a vaccinia virus vector or a sendai virus vector, an Epstein-Barr virus (EBV) vector, a HSV vector, or an mRNA transfection can be used. For example, a virus vector lacking the replicating ability so as not to self-replicate in an infected cell can be used. In other embodiments, a non-virus vector can also be used in the present disclosure in combination with a liposome or a condensing agent such as a cationic lipid as described in WO 96/10038 (which is incorporated herein by reference in its entirety), WO 97/18185 (which is incorporated herein by reference in its entirety), WO 97/25329 (which is incorporated herein by reference in its entirety), WO 97/30170 (which is incorporated herein by reference in its entirety), and WO 97/31934 (which is incorporated herein by reference in its entirety). The chimeric nucleic acid molecule can, in some embodiments, be also introduced into a cell by calcium phosphate transduction, DEAE-dextran, electroporation, or particle bombardment.

In yet other embodiments, when a retrovirus vector is used, the process of the present disclosure can be carried out by selecting a suitable packaging cell based on an LTR sequence and a packaging signal sequence possessed by the vector and preparing a retrovirus particle using the packaging cell. Examples of the packaging cell include PG13 (ATCC CRL 10686), PA317 (ATCC CRL-9078), GP+E-86 and GP+envAm-12 (U.S. Pat. No. 5,278,056, which is incorporated herein by reference in its entirety), and Psi-Crip. In certain examples. a retrovirus particle can also be prepared using a 293 cell or a 293T cell having high transfection efficiency.

In some embodiments, a method of producing a cell disclosed herein (e.g., a cell comprising a chimeric polypeptide, a chimeric nucleic acid molecule or both, such as, for example, a cell expressing the chimeric polypeptide) comprises adding a chimeric nucleic acid molecule or a vector described herein, to a cell. In other embodiments, a method of producing a cell herein disclosed (e.g., a cell comprising a chimeric polypeptide, a chimeric nucleic acid molecule or both, such as, for example, a cell expressing the chimeric polypeptide), comprises:
a) isolating a first cell (e.g., a cell disclosed here, such as, for example, an immune cell, T cell or NK cells (e.g., KHYG1, NK-92, YT, SNK-6, or NKL cells) from an animal (e.g., mammal, primate, or human));
b) adding a chimeric nucleic acid molecule (e.g., contained within a vector as disclosed herein) to the isolated first cell to produce a second cell (e.g., a cell comprising a chimeric polypeptide, a chimeric nucleic acid molecule or both, such as, for example, a cell expressing the chimeric polypeptide); and
c) optionally recovering and/or expanding the second cell.

In some embodiments, the first cell is an insect cell (e.g., an Sf9 cell), a vertebrate cell, or a mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, an adipose stem cell, an immune cell, a T cell, or an NK cell (e.g., KHYG1, NK-92, YT, SNK-6, or NKL cells)). In certain embodiments, the first cell can be any suitable cell including but not limited insect cell, a vertebrate cell, or a mammalian cell (e.g., a human cell, a rat cell a mouse cell, a NIH/3T3 cell, a CHO cell, a mesenchymal stem cell (MSC), a hematopoietic stem cell, a blood cell, a bone marrow cell, a stem cell, an adipose stem cell, an immune cell, a T cell, or an NK cell (e.g., KHYG1, NK-92, YT, SNK-6, or NKL cells)). In other embodiments, the first cell is autologous, allogeneic, a peripheral blood NK cell, a cord blood (allogeneic) NK cell, induced pluripotent stem cells, or an iPSC-derived NK cells (autologous or allogeneic). In certain embodiments, the first cell can be a cell from a mammal, a primate, a monkey, a human, a mouse, a rat, a pig, a horse, or a dog. In certain embodiments, the first cell can be collected, isolated, and/or purified from a body fluid, a tissue or an organ such as blood (e.g., peripheral blood or umbilical cord blood) or bone marrow or a cell obtained by differentiating or reprogramming the aforementioned cell to produce an induce pluripotent stem cell (iPSC) can be used. Any suitable cell can used for the first cell including but not limited to a peripheral blood mononuclear cell (PBMC), an immune cell (e.g., a T cell, a dendritic cell, a B cell, a hematopoietic stem cell, a macrophage, a monocyte, a NK cell (e.g., KHYG1, NK-92, YT, SNK-6, or NKL cells) or a hematopoietic cell (e.g., a neutrophil or a basophil), an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In certain embodiments, an NK cell (e.g., KHYG1, NK-92, YT, SNK-6, or NKL cells), a T cell, a precursor cell of a T cell (e.g., a hematopoietic stem cell or a lymphocyte precursor cell) or a cell population containing them can be used for the first cell. Any suitable T cell can be used for the first cell including but not limited a CD8-positive T cell, a CD4-positive T cell, a regulatory T cell, a cytotoxic T cell, and a tumor infiltrating lymphocyte. Any suitable NK cell can be used for the first cell including but not limited to KHYG1, NK-92, YT, SNK-6, or NKL cells. In certain embodiments, the first cell be collected from an animal (e.g., mammal, primate, or human), obtained by expansion culture of a cell collected from an animal (e.g., mammal, primate, or human), established as a cell strain, or a combination thereof. For example, when transplantation of the produced chimeric polypeptide-expressing cell or a cell differentiated from the produced chimeric polypeptide-expressing cell into an animal (e.g., mammal, primate, or human) may in some instances be desired, the chimeric nucleic acid molecule can be introduced into a cell collected from the an animal (e.g., mammal, primate, or human) itself or a conspecific an animal (e.g., mammal, primate, or human) thereof.

In some embodiments, the cells in step a are NK cells (e.g., KHYG1, NK-92, YT, SNK-6, or NKL cells). In some embodiments, the cells in step a are T cells. In other embodiments, the first cells in step a are CD3+, and optionally stimulated with an anti-CD3 antibody, optionally in a soluble or membrane-bound form (e.g., OKT3 or mOKT3), and/or APC prior to the adding. In other embodiments, the APC are artificial APC (aAPC). In another embodiment, the aAPC express a membranous form of anti-CD3 monoclonal antibody.

In certain embodiments, the adding step is repeated. In other embodiments, the adding step can be carried out 2 times, 3 times, 4 times, 5 times, 6 times, 7 times, 8 times or until in some instances an adequate level of chimeric polypeptide expression is achieved. In other embodiments, the adding step can be carried out five times. In some embodiments, if there are multiple adding steps, they can occur on one day or they can occur on more than one consecutive day. In some embodiments, if there are multiple adding steps, they can occur on two consecutive days, three consecutive days or four consecutive days.

In other embodiments, the second cells (e.g., a cell comprising a chimeric polypeptide, a chimeric nucleic acid molecule or both, such as, for example, a cell expressing the chimeric polypeptide) are stimulated with irradiated cells expressing a predetermined antigen (e.g., PD-1). In yet other embodiments, the cells are chimeric polypeptide expressing NK cells (e.g., KHYG1, NK-92, YT, SNK-6, or NKL cells) and are optionally stimulated with irradiated cells (e.g., expressing a predetermined antigen (e.g., PD-1)) at an effector to target ratio of 100:1, 75:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50, or 1:100.

### Compositions Including Pharmaceutical Compositions

One or more chimeric polypeptides, one or more chimeric nucleic acid molecules (e.g., in the form of a bare nucleic acid molecule, a vector, a virus, a plasmid or any suitable form), or one or more cells comprising a chimeric polypeptide, a chimeric nucleic acid molecule, or both, as disclosed herein, can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%. In certain embodiments, a chimeric polypeptide, chimeric nucleic acid molecule, or one or more cells comprising a chimeric polypeptide, a chimeric nucleic acid molecule, or both, can be part of the composition at any amount indicated herein (e.g., indicated above). In certain embodiments, cells (e.g., as disclosed herein) can be part of the composition in any suitable amount including but not limited to those indicated herein (e.g., indicated above).

One or more chimeric polypeptides, one or more chimeric nucleic acid molecules (e.g., in the form of a bare nucleic acid molecule, a vector, a virus, a plasmid or any suitable form), or one or more cells comprising a chimeric polypeptide, a chimeric nucleic acid molecule, or both, can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%. In some embodiments, isolated or purified means that impurities (e.g., cell components or unwanted solution components if chemically synthesized) are removed by one or more of any suitable technique (e.g., column chromatography, HPLC, centrifugation, fractionation, gel, precipitation, or salting out).

Some embodiments of the present invention include compositions comprising one or more chimeric polypeptides, one or more chimeric nucleic acid molecules (e.g., in the form of a bare nucleic acid molecule, a vector, a virus, a plasmid or any suitable form) or one or more cells comprising a chimeric polypeptide, a chimeric nucleic acid molecule, or both, as disclosed herein. In certain embodiments, the composition is a pharmaceutical composition, such as compositions that are suitable for administration to animals (e.g., mammals, primates, monkeys, humans, canine, porcine, mice, rabbits, or rats). In some embodiments, there may be inherent side effects (e.g., it may harm the patient or may be toxic or harmful to some degree in some patients).

In some embodiments, one or more chimeric polypeptides, one or more chimeric nucleic acid molecules (e.g., in the form of a bare nucleic acid molecule, a vector, a virus, a plasmid or any suitable form), or one or more cells comprising a chimeric polypeptide, a chimeric nucleic acid molecule, or both, can be part of a pharmaceutical composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In certain embodiments, a chimeric polypeptide, chimeric nucleic acid molecule (e.g., added as another vector or as part of the vector comprising chimeric nucleic acid), or one or more cells comprising a chimeric polypeptide, a chimeric nucleic acid molecule, or both, can be part of the pharmaceutical composition at any amount indicated herein (e.g., indicated above). In some embodiments, cells, such as cells comprising a chimeric polypeptide, a chimeric nucleic acid molecule, or both (e.g., as disclosed herein) can be part of the pharmaceutical composition at any suitable amount including but not limited to those indicated herein (e.g., indicated above).

In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, for the organ, or for the delivery route) including, but not limited to, suitable cell media, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. In some embodiments, the concentration of any individual formulary ingredient in a composition (e.g., pharmaceutical composition) can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the concentration of at least one formulary ingredient is not that same as that found in the natural system in which inventive polypeptide (e.g., chimeric polypeptide) is found. In some embodiments, the concentration of at least one formulary ingredient is not that same as that found in one or more natural systems (e.g., any natural system found in nature) in which the nucleic acid molecule which encodes an inventive polypeptide (e.g., chimeric polypeptide) is found.

In certain embodiments, pharmaceutical compositions can be formulated to release the active ingredient substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

### Methods to Treat Disease

Some embodiments of the invention include treatment of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) by administering one or more cells comprising a chimeric polypeptide, a chimeric nucleic acid molecule, or both, as disclosed herein, administering a composition as disclosed herein, or administering a pharmaceutical composition, as disclosed herein. One or more cells comprising a chimeric polypeptide, a chimeric nucleic acid molecule, or both, can be administered to animals by any number of suitable administration routes or formulations. One or more cells comprising a chimeric polypeptide, a chimeric nucleic acid molecule, or both, can also be used to treat animals for a variety of diseases. Animals include but are not limited to mammals, primates, rodents, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of cells comprising a chimeric polypeptide, a chimeric nucleic acid molecule, or both, can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intranasal administration, or intramuscular administration. The choice of administration route can depend on the cell identity (e.g., the strain, make-up, or physical and chemical properties of the cell) as well as the age and weight of the animal, the particular disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer), and the severity of the disease (e.g., stage or severity of disease). Of course, combinations of administration routes can be administered, as desired.

Some embodiments of the invention include a method for providing a subject with a composition comprising one or more cells comprising a chimeric polypeptide, a chimeric nucleic acid molecule, or both, which comprises one or more administrations of one or more such compositions; the compositions may be the same or different if there is more than one administration.

In some embodiments, diseases that can be treated in an animal (e.g., mammals, porcine, canine, avian (e.g., chicken), bovine, feline, primates, rodents, monkeys, rabbits, mice, rats, and humans) using an one or more cells comprising a chimeric polypeptide, a chimeric nucleic acid molecule, or both, include, but are not limited to autoimmune disease, allergies (e.g., IgE related allergies), asthma, Sjögren's syndrome, juvenile dermatomyositis, multiple sclerosis, type-1 diabetes mellitus, rheumatoid arthritis, systemic lupus erythematosus (SLE), cancer, blood cancer, solid tumor, leukemia, lymphoma, myeloma, breast cancer, ovarian cancer, glioblastoma, osteosarcoma, medulloblastoma, inflammatory disease, eczema, hepatitis, or an infectious disease (e.g., as caused by a virus such as influenza and HIV, a bacterium, or a fungus, for example, tuberculosis, MRSA, VRE, and deep mycosis). In other embodiments, diseases that can be treated using one or more cells comprising a chimeric polypeptide, a chimeric nucleic acid molecule, or both, include, but are not limited to autoimmune disease, Sjögren's syndrome, juvenile dermatomyositis, multiple sclerosis, type-1 diabetes mellitus, rheumatoid arthritis, systemic lupus erythematosus (SLE), or cancer. In certain embodiments, diseases that can be treated can include autoimmune disease, allergies (e.g., IgE related allergies), asthma, Sjögren's syndrome, juvenile dermatomyositis, multiple sclerosis, type-1 diabetes mellitus, rheumatoid arthritis, or systemic lupus erythematosus (SLE). In certain embodiments, diseases that can be treated can include autoimmune disease or systemic lupus erythematosus (SLE).

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease).

In some embodiments, diseases (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) that can be treated in an animal (e.g., mammals, porcine, canine, avian (e.g., chicken), bovine, feline, primates, rodents, monkeys, rabbits, mice, rats, and humans) using one or more cells comprising a chimeric polypeptide, a chimeric nucleic acid molecule, or both, include, but are not limited to diseases (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) that can be treated by targeting T_{FH}, killing T_{FH}, binding to PD-1 (e.g., human PD-1, mouse PD-1, rat PD-1, monkey PD-1, mammal PD-1, primate PD-1, or SEQ ID NO:34) on another cell, killing T_{FR} cells (follicular regulatory T cells), killing activated T cells, killing activated B cells, killing CD80 expressing cells, killing PD-1 expressing cells, killing natural killer T cells, killing CD8 expressing cells, killing CD4 expressing T cells, or inducing degranulation, or a combination thereof.

In certain embodiments, one or more cells comprising a chimeric polypeptide, a chimeric nucleic acid molecule, or both, can be targeted or localized to certain tissues or organs. In other embodiments, one or more cells comprising a chimeric polypeptide, a chimeric nucleic acid molecule, or both, can be targeted or localized to follicular regions of secondary lymphoid tissues (e.g., by expressing of CXCR5 in the cell, by, for example, transfection or transduction).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating diseases (e.g., autoimmune disease, Sjögren's syndrome, juvenile dermatomyositis, multiple sclerosis, type-1 diabetes mellitus, rheumatoid arthritis, systemic lupus erythematosus (SLE), or cancer), treating can include but is not limited to prophylactic treatment and therapeutic treatment. As such, treatment can include, but is not limited to: preventing disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer); ameliorating or relieving symptoms of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer); inhibiting the development or progression of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer); reducing the severity of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) or one or more of the symptoms associated with disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) (e.g., a decrease in the amount of T_{FH}); or causing remission of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer). In some embodiments, treating does not include prophylactic treatment of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) (e.g., preventing or ameliorating future disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer)).

Treatment of an animal (e.g., human) can occur using any suitable administration method (such as those disclosed herein) and using any suitable amount of one or more cells comprising a chimeric polypeptide, a chimeric nucleic acid molecule, or both. In some embodiments, methods of treatment comprise treating an animal for disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer). Some embodiments of the invention include a method for treating a subject (e.g., an animal such as a human or primate) with a composition comprising one or more cells comprising a chimeric polypeptide, a chimeric nucleic acid molecule, or both (e.g., a pharmaceutical composition) which comprises one or more administrations of one or more such compositions; the compositions may be the same or different if there is more than one administration.

In some embodiments, the method of treatment includes administering an effective amount of a composition comprising one or more cells comprising a chimeric polypeptide, a chimeric nucleic acid molecule, or both. As used herein, the term “effective amount” refers to a dosage or a series of dosages sufficient to affect treatment (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of one or more cells comprising a chimeric polypeptide, a chimeric nucleic acid molecule, or both (which can be administered to an animal such as mammals, primates, monkeys or humans) can be an amount of about 0.005 to about 50 mg/kg body weight, about 0.005 to about 80 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, an effective amount of one or more cells comprising a chimeric polypeptide, a chimeric nucleic acid molecule, or both (which can be administered to an animal such as mammals, rodents, mice, rabbits, feline, porcine, or canine) can be an amount of about 0.005 to about 50 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of one or more cells comprising a chimeric polypeptide, a chimeric nucleic acid molecule, or both (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 5 mg/kg human body weight, about 10 mg/kg human body weight, about 20 mg/kg human body weight, about 80 mg/kg human body weight, or about 100 mg/kg human body weight. In some instances, an effective amount of one or more cells comprising a chimeric polypeptide, a chimeric nucleic acid molecule, or both (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 80 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

“Therapeutically effective amount” means an amount effective to achieve a desired and/or beneficial effect (e.g., decreasing T_{FH}). A therapeutically effective amount can be administered in one or more administrations. For some purposes of this invention, a therapeutically effective amount is an amount appropriate to treat an indication (e.g., to treat disease, such as for example, autoimmune disease, Sjögren's syndrome, juvenile dermatomyositis, multiple sclerosis, type-1 diabetes mellitus, rheumatoid arthritis, systemic lupus erythematosus (SLE), or cancer). By treating an indication is meant achieving any desirable effect, such as one or more of palliate, ameliorate, stabilize, reverse, slow, or delay disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) progression, increase the quality of life, or to prolong life. Such achievement can be measured by any suitable method, such as but not limited to measurement of the amount of T_{FH} cells in a subject, decrease in fever, decrease of facial rash, or the extent of cancer growth or of metastasis.

In some embodiments, treatment of a disease can be a result of the activation of a cell comprising a chimeric polypeptide, a chimeric nucleic acid molecule, or both. In certain embodiments, activation (e.g., and certain forms or types of activation) of such a cell can depend on the type of cell (e.g., T cell vs. NK cell) and the specific intracellular segment of the chimeric polypeptide. In other embodiments, activation can result in one or more of release of a cytokine (e.g., cytotoxic cytokine), degranulation, improvement of cell proliferation rate, or change in a cell surface molecule. In yet other embodiments, release of a cytotoxic cytokine (e.g., a tumor necrosis factor or a lymphotoxin) from the activated cell can cause destruction of a target cell (e.g., T_{FH}) expressing an antigen (e.g., PD-1). In still other embodiments, release of a cytokine or change in a cell surface molecule can stimulate other immune cells (e.g., B cell, dendritic cell, NK cell, or a macrophage), which can, in some instances, assist in the treatment of the disease.

In some embodiments, other treatments of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) are optionally included, and can be used with the inventive treatments described herein (e.g., administering one or more one or more cells comprising a chimeric polypeptide, a chimeric nucleic acid molecule, or both, as disclosed herein,)). Other treatments of disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer) can include any known treatment that is suitable to treat the disease (e.g., autoimmune disease, Sjögren's syndrome, juvenile dermatomyositis, multiple sclerosis, type-1 diabetes mellitus, rheumatoid arthritis, systemic lupus erythematosus (SLE), or cancer).

In some embodiments, additional optional treatments (e.g., as an other disease treatment) can also include one or more of surgical intervention, hormone therapies, immunotherapy, administration of NSAIDs, administration of immune-suppressing drugs, and adjuvant systematic therapies.

The presently-disclosed subject matter is further illustrated by the following specific but non-limiting examples. The following examples may include compilations of data that are representative of data gathered at various times during the course of development and experimentation related to the present invention.

### Examples

### Materials and Methods

### Cell Culture, Microscopy, & Counting

The NK-92 (ATCC CRL-2407™) and Raji (ATCC CCL-86™) cell lines were obtained from ATCC and cultured according to their standard protocols using the appropriate media as described on the ATCC website. S2 cells were obtained from ThermoFisher (R69007) and cultured according to their standard protocol (MARCH et al. (2010) “Use of transfected Drosophila S2 cells to study NK cell activation” Methods Mol. Biol., Vol. 612, pp. 67-88, which is incorporated herein by reference in its entirety) in Schneider's Drosophila Media (ThermoFisher) containing 10% heat-inactivated fetal bovine serum (HI-FBS, Gibco). When necessary for selection, blasticidin or puromycin were added at a concentration of 25 μg/mL and 2 μg/mL, respectively. All cultures involving primary tonsillar lymphocytes were performed in RPMI-1640 (HyClone SH30255) supplemented with 10% HI-FBS, 100U/mL penicillin, 100 μg/mL streptomycin, 1 mM sodium pyruvate, 10 mM HEPES buffer, 1×MEM non-essential amino acids, and 0.1 mM 2-beta-mercaptoethanol (henceforth referred to as “cRPMI”).

Fluorescent cell images were taken using a Nikon Eclipse Ti microscope, equipped with Zyla sCMOS camera (Andor) and 488 nm filter cube, and processed using NIS-Elements Imaging software (Nikon). All cell enumerations were obtained with a hemocytometer using Trypan Blue exclusion.

### Collection & Processing of Human Tissue Samples

### Study Participants

Male and female children 9 years old or younger requiring tonsillectomy were recruited to a prospective study at a tertiary academic care center through the Division of Pediatric Otolaryngology-Head and Neck Surgery at the Cincinnati Children's Hospital Medical Center (CCHMC). Criteria for enrollment in the study included a history of sleep-disordered breathing or recurrent or chronic tonsillitis requiring removal of the tonsillar tissue. Consent was obtained from parents in the perioperative suite on the day of the procedure. Children were excluded from the study if the tonsillar tissue was acutely infected or if anatomic abnormalities were present requiring a more detailed pathologic evaluation. Institutional review board (IRB) approval at CCHMC was obtained prior to initiation of this project.

### Tonsillar Tissue

After recruitment, patients underwent tonsillectomy as part of the standard of practice. After removal, approximately ½ of each of the bilateral tonsils were transected and placed in RPMI-1640 media with 10% human AB serum. Samples were labeled with a deidentified barcode and transferred to the research team for further processing. The remaining tonsillar tissue was sent to pathology for gross evaluation as part of the routine clinical care.

Tonsil tissue was processed via mincing with scissors followed by transfer of up to 4 g of tissue to a gentleMACS C tube (Miltenyi Biotec) containing 8 mL of phosphate-buffered saline (PBS) with 0.5 mg/mL collagenase D and 3000 U/mL DNaseI, then dissociated on a GentleMACS Octo Dissociator (Miltenyi Biotec) using “program C”. Homogenates were incubated in a 37° C. water bath for 15 minutes, then dissociated again using “program C” and transferred through a 100 um cell strainer into cRPMI. This cell suspension was then layered over Ficoll-Paque PLUS (GE Healthcare) and subjected to density-gradient centrifugation to isolate tonsillar mononuclear cells.

### Peripheral Blood Mononuclear Cells (PBMC)

PBMC were obtained by thorough PBS-washing of deidentified leukocyte reduction filters obtained from the University of Cincinnati Hoxworth Blood Center with IRB approval at CCHMC. Washed filter cells were then subjected to density-gradient centrifugation over Ficoll-Paque PLUS for PBMC isolation.

All PBMC and tonsil mononuclear cells were cryopreserved in FBS containing 10% dimethyl sulfoxide and frozen @−1° C./min using CoolCell freezing containers to −80° C., then stored long-term in liquid nitrogen. Cryopreserved cells were rapidly thawed and transferred into cRPMI before use.

### Flow Cytometry & Cell Sorting

Fluorescently-conjugated antibodies used were obtained from either Biolegend, Ebioscience (ThermoFisher), Invitrogen, or BD Biosciences.

For surface staining, cells were resuspended at a concentration of 1-2×10⁶/mL in 50-100 μL of cold Hank's Buffered Salt Solution (HBSS) containing 5% HI-FBS, 100U/mL penicillin, and 100 μg/mL streptomycin, (henceforth referred to as “FACS buffer”). Fluorescently-conjugated antibodies were each added at the manufacturer's recommended concentration. Cells were incubated at 4° C. for 20 minutes, then washed twice with FACS buffer, and either analyzed fresh (same-day), fixed using 100 μL BD Cytofix for 20 minutes at 4 C, or stained for intracellular markers. Intracellular staining was performed by fixation/permeabilization of surface-stained cells in 100 μL Cytofix/Cytoperm (BD Biosciences) for 20 minutes at 4° C., followed by staining in 100 μL 1× Perm/Wash buffer (BD Biosciences) containing fluorescently-conjugated antibodies, each at the manufacturer's recommended concentration, for 20 minutes at 4° C. All fixed cells were washed twice with FACS buffer to remove fixative, and kept at 4° C. in FACS buffer until analysis (1-3 days later).

Acquisition of stained cells was performed using a Fortessa or LSRII cytometer (BD Biosciences) with FACSDiva software (BD Biosciences). Flow cytometric cell sorting was performed using an SH800S cell sorter (Sony) with the accompanying Sony acquisition software.

### Design & Generation of PD-L1 CAR Vector & PD-1-Containing Vectors

As used herein CAR is an example of a chimeric polypeptide and the CAR's encoding nucleic acid is an example of a chimeric nucleic acid molecule.

In general, plasmid amplifications were performed by transformation and expansion of the competent E. coli strains DH5α or XL10-Gold (for pUC57 plasmids), and Stbl3 or StellarComp (for pLVX-IRES-ZsGreen1, PiggyBac, and pAc/V5-His plasmids), grown in LB broth/agar plates containing the appropriate selective antibiotic (50 μg/mL ampicillin or 100 μg/mL kanamycin). Digestions were performed according to the relevant New England BioLabs online NEBcloner protocol. Digestion products were resolved using 1% agarose gel electrophoresis. DNA-containing gel fragments were purified using the GeneJET Gel Extraction kit (ThermoFisher). Ligations were performed for two hours at room temperature using T4 DNA ligase (ThermoFisher). Final plasmids were purified using QIAprep Mini, Midi, or Maxi kits (Qiagen) and stored at −20° C. Plasmid insertions were verified via Sanger sequencing using custom primers (IDT Technologies or Invitrogen). Sequence files were aligned using Snapgene and/or Benchling.

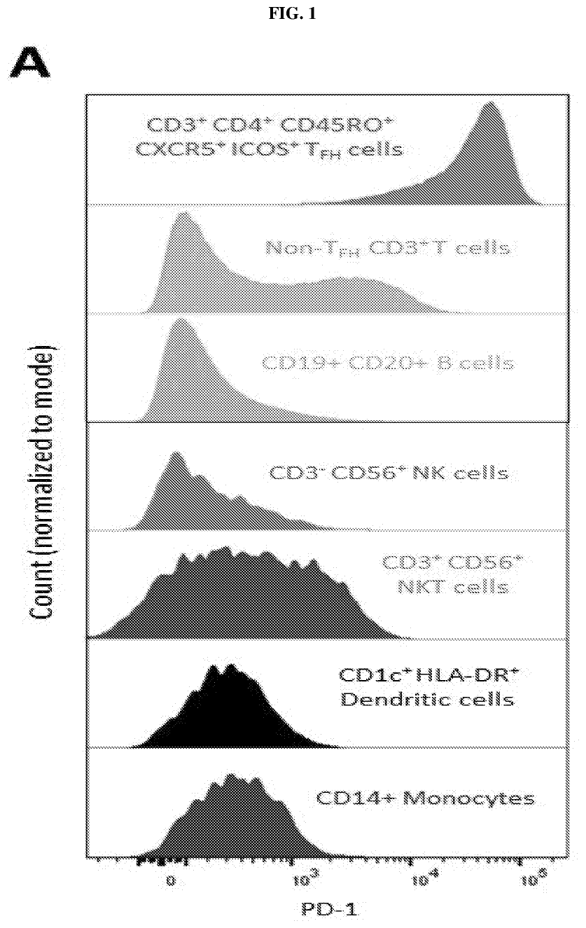

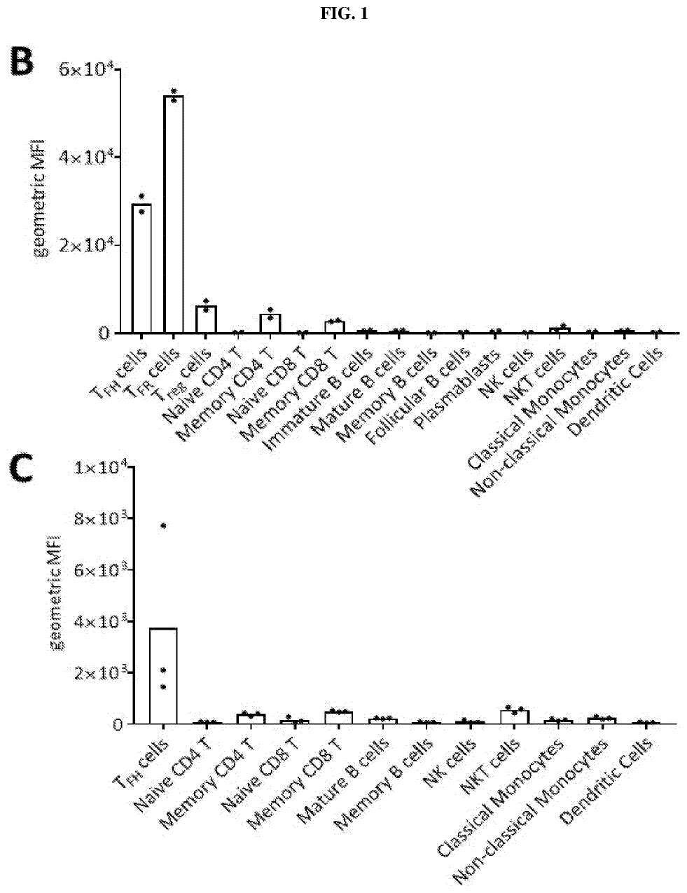

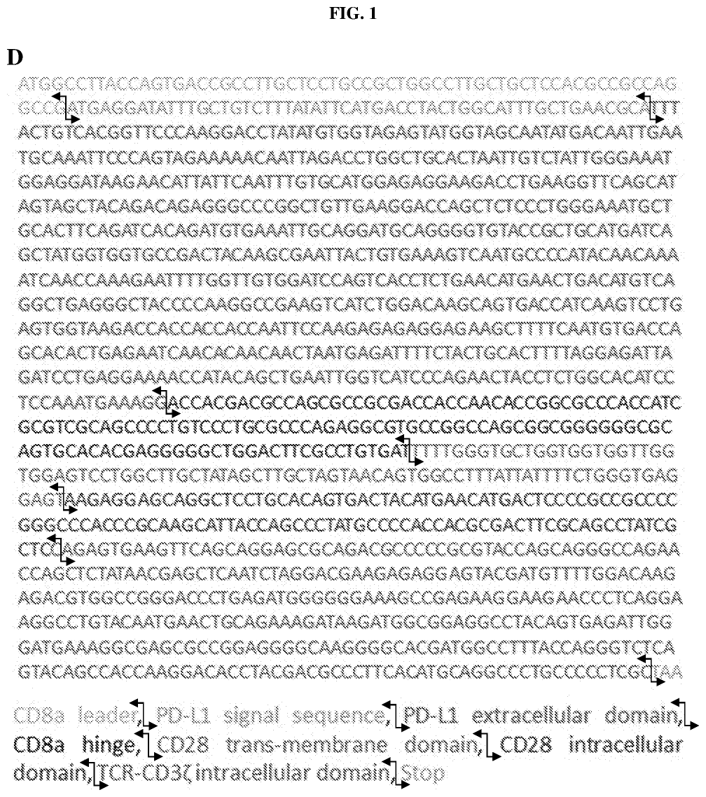

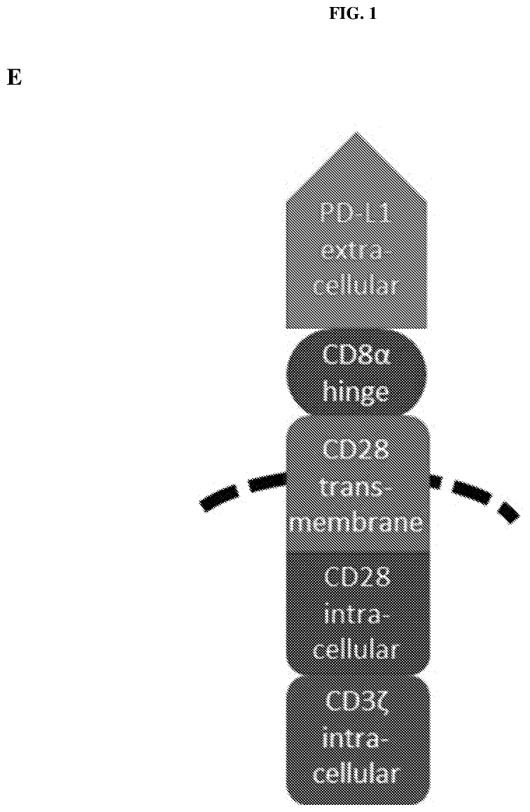

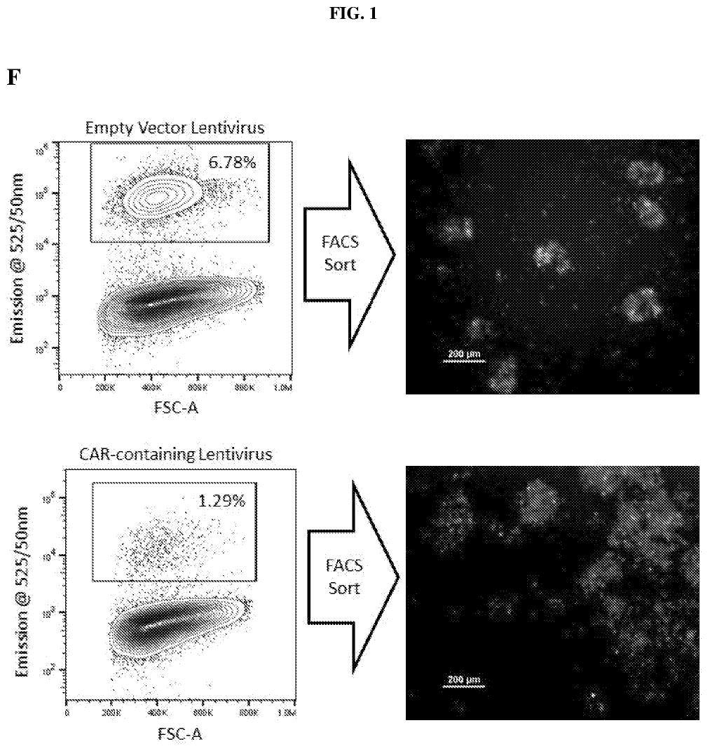

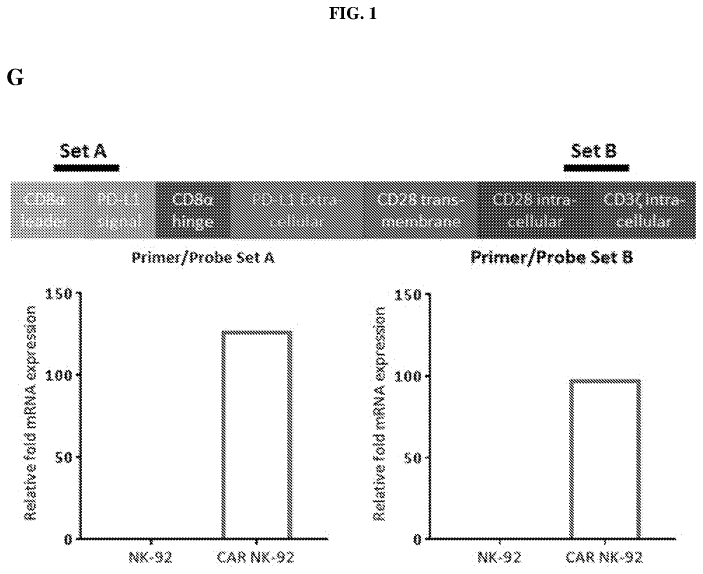

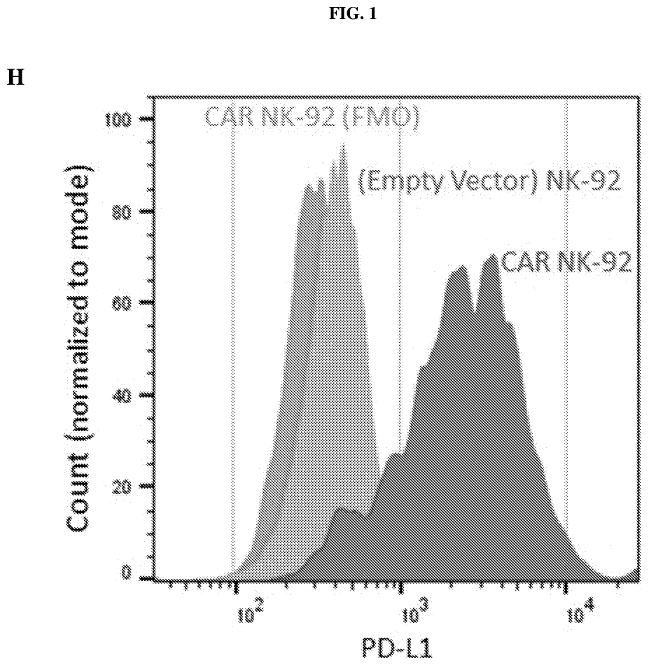

The 2^{nd} generation CAR (PDL1-CD28-CD3ζ) was designed by splicing the PD-L1 signal and extracellular sequences (amino acid 1-238, NP_054862, SEQ ID NO:6, and nucleic acid sequence is SEQ ID NO:33; the full amino acid sequences for PD-L1 is SEQ ID NO:38) to typical 2^{nd} generation CAR sequences (GUEDAN et al. (2019) “Engineering and design of chimeric antigen receptors” Mol. Ther. Methods Clin. Dev., Vol. 12, pp. 145-146, which is incorporated herein by reference in its entirety; HAN et al. (2015) “CAR-engineered NK cells targeting wild-type EGFR and EGFRvIII enhance killing of glioblastoma and patient-derived glioblastoma stem cells” Sci. Rep., Vol. 5, pp. 1-13, which is incorporated herein by reference in its entirety; SUN et al. (2014) “Construction and evaluation of a novel humanized HER2-specific chimeric receptor” Breast Cancer Res., Vol. 16, Article R61, 10 pages, which is incorporated herein by reference in its entirety), including the leader and hinge regions of CD8α, CD28 transmembrane & intracellular sequences, and CD3ζ intracellular sequences (FIGS. 1D and 1E).

The CAR sequence was synthesized by Genewiz into a pUC57 vector. The CAR construct was excised from pUC57 and ligated into the multiple cloning site (MCS) of the lentiviral vector pLVX-IRES-ZsGreen1 (Clontech) (SEQ ID NO:36). Clones were screened for the correct restriction digestion pattern and sequence-verified (see primers in SEQ ID NO:40, SEQ ID NO:41, and SEQ ID NO:42) prior to being amplified and purified.

The full length, human PD-1 protein sequence was obtained from UniProt (a.a. 1-288) (SEQ ID NO:34). An open reading frame (ORF) for this amino acid sequence was optimized for a Drosophila expression system using the “codon optimization tool” (IDT Technologies) (SEQ ID NO:35), then this DNA sequence was synthesized by Genewiz into a Puc57 plasmid. The PD-1 ORF was excised from pUC57 and ligated into the MCS of a pAc/V5-His A vector (Invitrogen), then verified via sequencing (see primers in SEQ ID NO:43 and SEQ ID NO:44), amplified, and purified (referred to as “pAc/V5-His-PD1”—SEQ ID NO:37).

The human PD-1 ORF was excised from the aforementioned pUC57 plasmid using and ligated into the MCS of a PiggyBac Transposon, Cloning and Expression Vector (System Biosciences, PB513B-1), then amplified, purified, and verified (see primers in SEQ ID NO:45 and SEQ ID NO:46) via sequencing (referred to as “PB513-PD1”—SEQ ID NO:39).

### Lentiviral Generation and Transduction of NK-92 Cells

After sequence verification the CAR-containing pLVX-IRES-ZsGreen plasmid was given to the Cincinnati Children's Viral Vector Core for lentivirus production. Briefly, lentivirus was packaged by transfection of 293-T cells with a 3rd generation packaging system: pCDNA3.g/p.4×CTE plasmid (GagPol, 8 μg/plate), pRSV rev plasmid (Rev, 6.5 μg/plate), vector plasmid (8 μg/plate), and m75-VSVG plasmid (VSV-G envelope, 2 μg/plate). Viral supernatant from four 10-cm plates was collected 24-48 hours post-transfection, purified via sucrose-gradient, and titer analysis was performed. Viral supernatant was concentrated to 350 μL and stored at −80° C. in 25 μL aliquots. Titer was determined by transfection of control cell line and flow cytometry analysis.

NK-92 were transduced in 48 or 96-well flat bottom plates, previously coated overnight with human fibronectin at 20 μg/mL. Cells were transduced with either CAR-containing pLVX-IRES-ZsGreen lentiviral vector or an empty-vector control lentivirus at a multiplicity of infection of 5 in 8 μg/mL protamine sulfate for 4-6 hours at 37° C. After 48 hours, cells were sorted based on fluorescent reporter expression.

### Generation of PD-1-Expressing Target Cell Lines

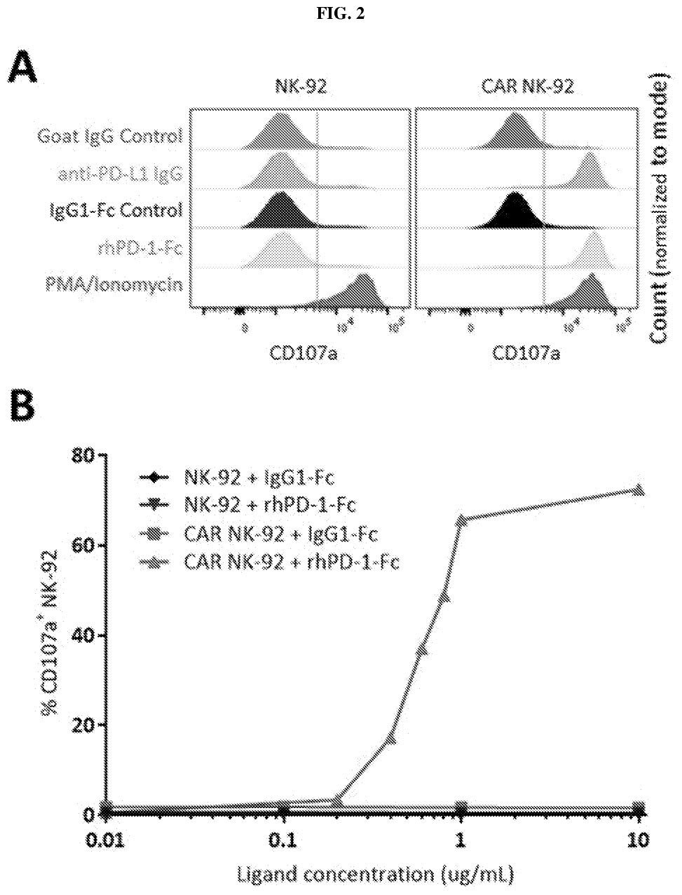

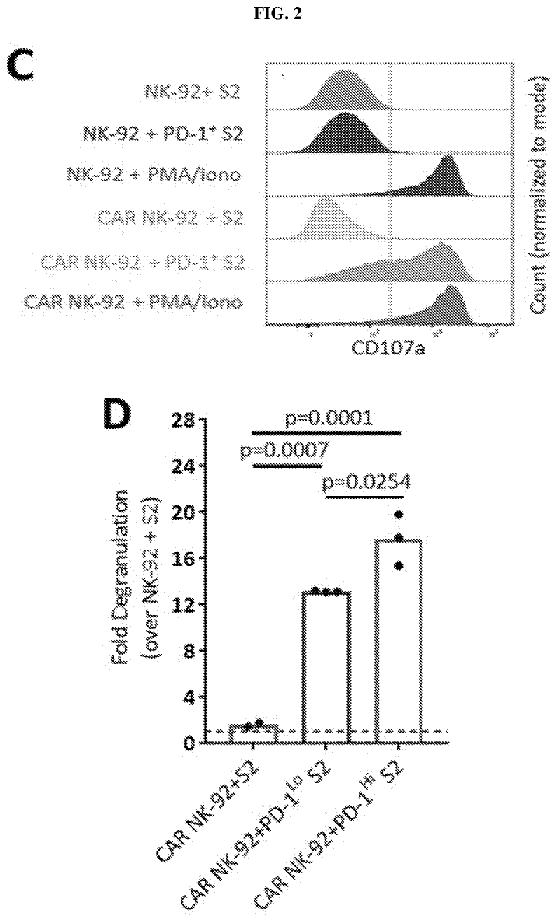

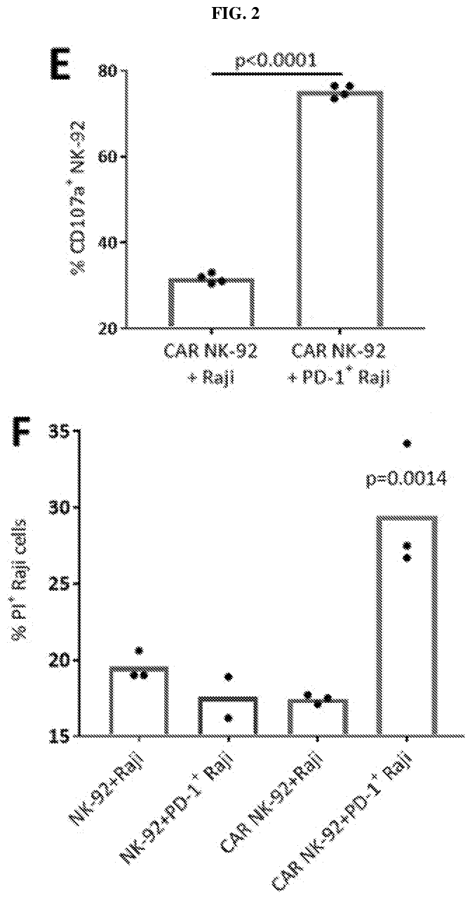

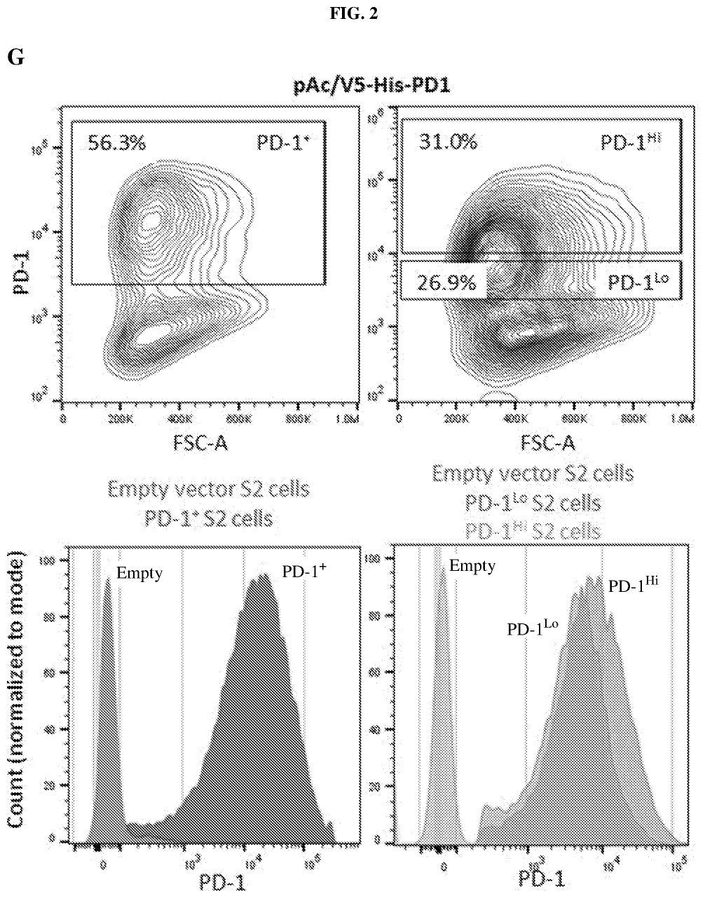

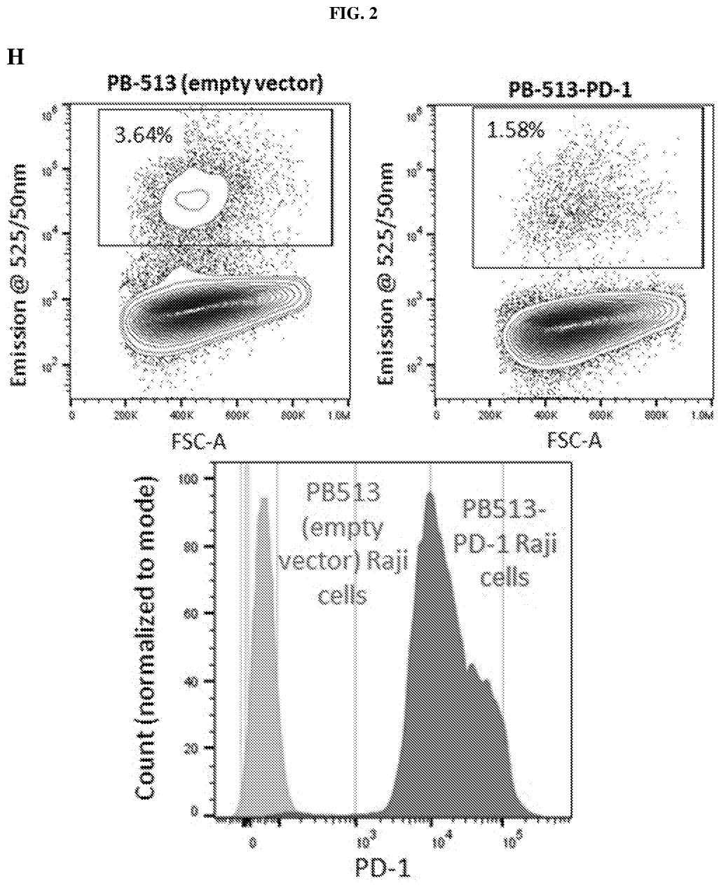

S2 cells were chemically transfected (CaCl₂)) using the DES Blasticidin Support Kit (Invitrogen) protocol. Briefly, the pAc/V5-His-PD1 plasmid (or empty-vector control plasmid) was introduced at a ratio of ≥19:1 to the pCoBlast plasmid vector (Invitrogen). Cells were cultured for a week in the presence of blasticidin to select for pCoBlast-expressing clones. Blasticidin-resistant cells transfected with pAc/V5-His-PD1 were stained for PD-1 expression and sorted for PD-1+ cells (see FIG. 2G). Following >2 weeks of culture, a portion of PD-1+S2 cells were stained for PD-1 expression and further sorted into “PD-1^{Hi}” and “PD-1^{Lo}” populations (see FIG. 2G). All sorted S2 cells were cultured for >2 weeks prior to co-culture with NK-92 to minimize any effects of residual αPD-1 antibody still bound to S2 cell surfaces.

Raji cells were co-transfected via electroporation with either the empty PiggyBac plasmid (PB513) or PB513-PD1 in addition to the Super PiggyBac transposase plasmid (System Biosciences, PB210PA-1) at a 1:2.5 ratio using the Neon Transfection System (Invitrogen, MPK5000). Electroporation parameters and cell concentration were obtained from the Neon Transfection System standard protocol for Raji cells (using 100 μL tips). Transfected Raji cells were cultured in 6-well, low adherent plates prior to fluorescent sorting and subsequent puromycin selection.

qPCR

NK-92 total RNA was extracted per RNeasy Mini Kit manufacturing protocol (Qiagen). Reverse transcription was performed using iScript Reverse Transcriptase and iScript Reaction mix per iScript cDNA Synthesis Kit manufacturing protocol (Bio-Rad). Real-Time PCR reactions were carried out using a PrimeTime Gene Expression Master Mix and custom PrimeTime Std qPCR assay primer/probe sets (IDT Technologies)(see Table 1), as well as a TaqMan Gene Expression Assay (ThermoFisher). An ABI 7500 Real-Time PCR Thermal Cycler (ThermoFisher) was used under the PCR polymerase activation and amplification conditions of 95° C. for 3 minutes and 40 cycles (95° C. for 15s and 60° C. for 1 minute).

TABLE 1

| | |
|---|---|
| PD-L1 CAR Set | 5′-/56-FAM/ATCGCTCCA/ZEN/GAGTGAAGTTCAGCA/3IABKFQ/-3′ |
| A-Probe | |
| PD-L1 CAR Set | GCAAGCATTACCAGCCCTAT (SEQ ID NO: 47) |
| A-Primer 1 | |
| PD-L1 CAR Set | TTCTGGCCCTGCTGGTA (SEQ ID NO: 48) |
| A-Primer 2 | |
| PD-L1 CAR Set | 5′-/56-FAM/CCAGGCCGA/ZEN/TGAGGATATTTGCTGT/3IABKFQ/-3′ |
| B-Probe | |
| PD-L1 CAR Set | CTTACCAGTGACCGCCTTG (SEQ ID NO: 49) |
| B-Primer 1 | |
| PD-L1 CAR Set | CTTGGGAACCGTGACAGTAAA (SEQ ID NO: 50) |
| B-Primer 2 | |

### Plate-Bound Activation Assay

rhPD-1-Fc, anti-PD-L1, IgG1-Fc, and Goat IgG (R&D systems) were solubilized in PBS and added at varying concentrations in 100 μL/well to 96-well MaxiSorp plates (ThermoFisher) before overnight incubation at 4° C. Plates were washed with PBS before counted NK-92 cells were added at a concentration of 4×10⁴ cells/mL in 200 μL per well and incubated for 4 hours at 37° C. Degranulation was measured via addition of 2 μg/mL of anti-CD107a PE-Cy7 (Biolegend) for the duration of the assay. PMA and ionomycin were added at 1 μg/mL each for the duration of the assay.

### NK-92 Co-Culture Activation & Cytotoxicity Assays

For NK-92:S2 co-culture assays, NK-92 and S2 cells were combined at a ratio of 1:5 (2×10⁴ and 1×10⁵, respectively) in 200 μL of S2 media (without blasticidin) in 96-well round-bottom plates, then incubated at room temperature for 4 hours prior to analysis.

For NK-92:Raji co-culture assays, NK-92 and Raji cells were combined at either a ratio of 1:5 (2×10⁵ and 1×10⁵, respectively) to assess degranulation, or a ratio of 20:1 (2×10⁵ and 1×10⁴, respectively) to assess killing. Assays were carried out in in 200 μL of Raji media (without puromycin) in 96-well round-bottom plates, then incubated at 37° C. for 4 hours prior to analysis.

For NK-92:CD4 co-culture assays, human tonsil cells were subjected to CD4 T cell negative magnetic isolation (StemCell Technologies), achieving 88% purity, on average (data not shown), then counted. This isolated CD4 fraction was counted and cultured in 96 well round-bottom plates at 30,000 cells/well in 100 μL of cRPMI. 1.5×10⁵ control or CAR NK-92 were added in 100 ml cRPMI to each well for a 5:1 ratio of NK:T cells, whereas control wells received 100 μL of cRPMI alone. Cells were then incubated for 4 hours at 37° C. prior to collection and analysis.

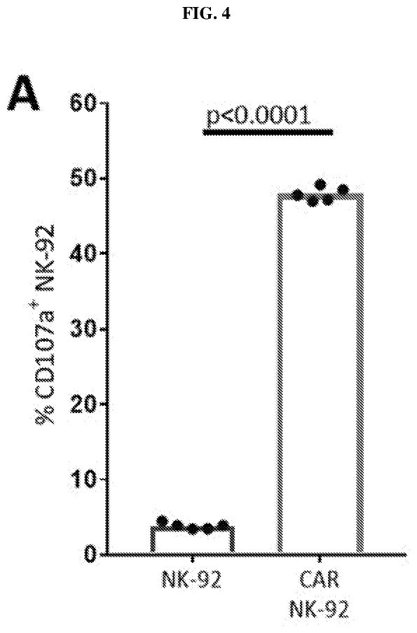

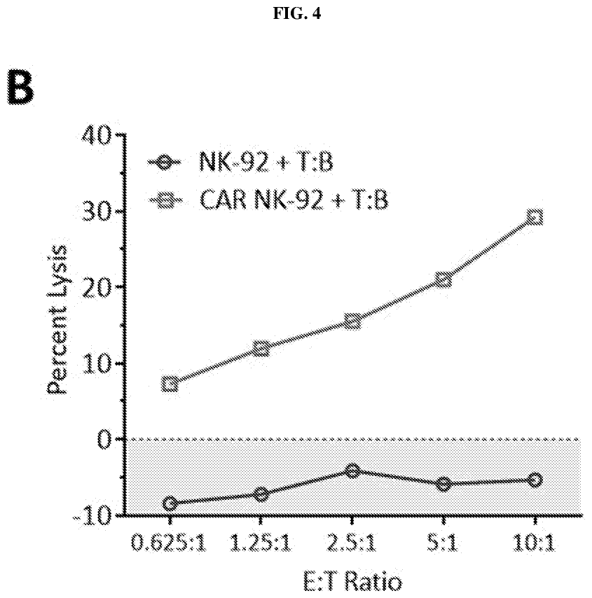

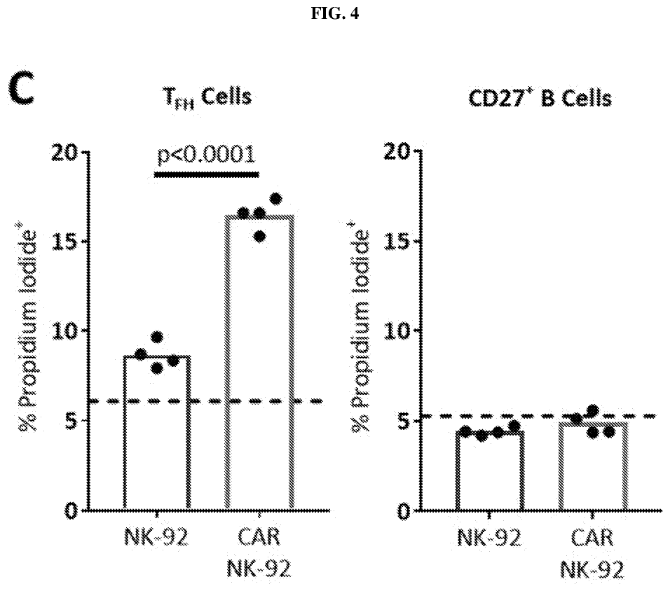

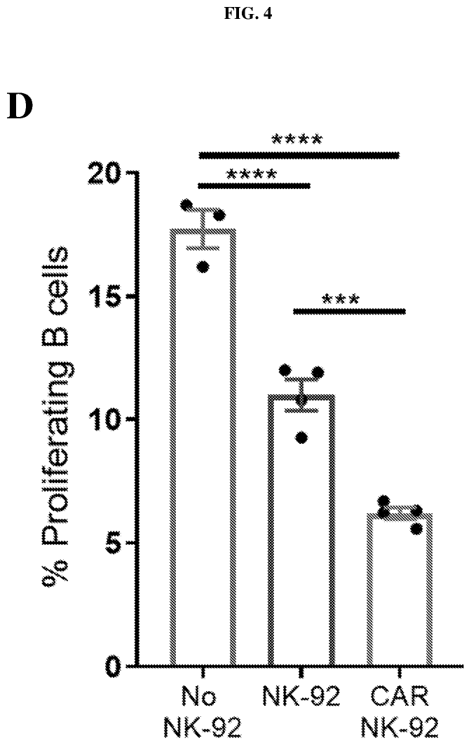

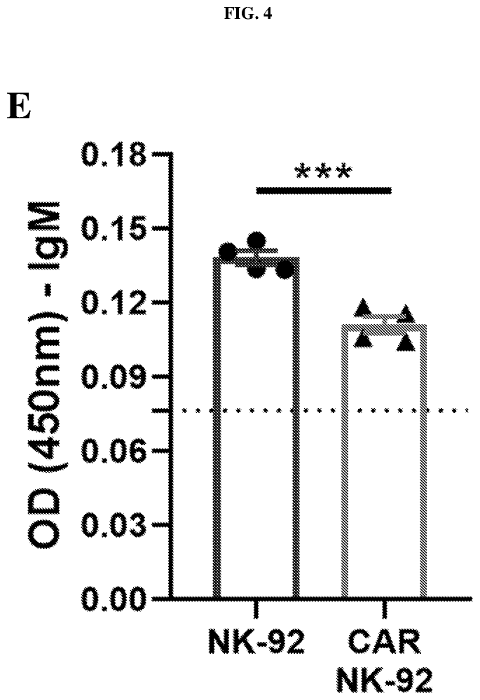

For NK-92: T_{FH}:B cell co-culture assays in FIGS. 4A-C, human tonsil cells were subjected to either CD4 T cell or B cell negative magnetic isolations (StemCell Technologies), achieving >80% purity (data not shown). The isolated CD4 T cell fraction was stained with fluorescently-conjugated antibodies to identify and sort for live (Zombie⁻) CD3⁺ CD4⁺ CXCR5⁺ T_{FH} cells. Similarly, isolated and stained B cells were sorted to obtain live (Zombie⁻) CD3⁻ C19⁺ CD27⁺ memory B cells. Sorted T_{FH} and B cells were cultured separately overnight (at 37° C.) in 96 well round-bottom plates at 3×10⁴ cells/well and 6×10⁴ cells/well, respectively, in 100 μL of cRPMI containing 1 μg/mL SEB (EMD Millipore). The next day, each well of T_{FH} cells was combined with a single well of memory B cells (achieving a 1:2 ratio of T:B cells) and further cultured. At days 3-4 post initial cell culture, 1.5×10⁵ control or CAR NK-92 were added in 50 ml cRPMI to each well to achieve a 5:1 ratio of NK:T_{FH} cells, whereas control wells received 50 μL of cRPMI alone. Co-cultures were then incubated for 4 hours at 37° C. prior to collection and analysis.

For FIGS. 4D-E, human tonsil cells were subjected to CD4 T cell negative magnetic isolation (StemCell Technologies), achieving 88% purity, on average (data not shown), then counted. This CD4 fraction was cultured in 96 well round-bottom plates at 30,000 cells/well in 100 μL of complete media. 1.5×10⁵ control or CAR NK-92 were added in 100 ml complete media to each well for a 5:1 ratio of NK:T cells, whereas control wells received 100 μL of media alone. Cells were incubated for 4 hours at 37° C. prior to collection and magnetic depletion of NK-92 cells using anti-human CD56 MicroBeads and LS separation columns (Miltenyi Biotec). The depletion of NK-92 (>99.9%) was verified by flow cytometry (data not shown). In parallel, human tonsil cells were subjected to B cell negative magnetic isolation (StemCell Technologies), then labeled with CellTrace Violet (Thermo Fisher) according to manufacturer's instructions. NK-92-depleted CD4 T cells were added to CellTrace-labeled B cells at a 1:2 ratio of T:B cells and cultured in complete media containing 1 μg/mL SEB (EMD Millipore) in 96 well round-bottom plates for 5 days. Supernatants were collected and tested for the presence of human IgM using a Human IgM ELISA kit (Thermo Fisher). At day 5, cells were collected and stained, and B cell proliferation was indicated by loss of CellTrace expression when examined via flow cytometry.

For calcein AM release assays, sorted T_{FH} and CD27+ B cell co-cultures (at a 1:2 T:B ratio; previously stimulated with SEB for 4 days as described above) were labeled with 1:300 dilution (volume:volume) of 1 mg/mL Calcein AM solution according a published protocol (SOMANCHI et al. (2011) “Expansion, purification, and functional assessment of human peripheral blood NK cells” J. Vis. Exp., Vol. 48, Article 2540, 5 pages, which is incorporated herein by reference in its entirety). Labeled cells were transferred to 96 well round-bottom plates at 1×10⁴ cells/well in 100 μL cRPMI. Control or CAR NK-92 were added in 100 ml cRPMI to wells at varying E:T ratios, whereas control wells received 100 μL of cRPMI alone. These co-cultures were carried out for 4 hours at 37° C. prior to collection and analysis.

When measured, degranulation was assessed by addition of 2 μg/mL of anti-CD107a PE-Cy7 (Biolegend) for the duration of the assay. When used, PMA and ionomycin were added at 1 μg/mL each for the duration of the assay. When measured, propidium iodide (Biolegend) was added at a 1:10 ratio (volume:volume) with stained, unfixed cells approximately 10-15 minutes prior to cytometric acquisition.

### Statistical Analysis

All flow cytometric analysis was performed using FlowJo v.10 software. Compensation was performed using single-color-stained cells and/or beads (ThermoFisher), and all electronic gating was performed downstream of a FSC-H×FSC-A “singlet” gate.

All statistical analysis was performed using GraphPad Prism 7. Data in FIGS. 2E, 3B, 4A, and 4C were each analyzed via an unpaired, two-tailed, parametric Student's t test with a 95% confidence interval (due to normal distribution). Due to comparisons across more than two conditions, data in FIGS. 2D and 2F were analyzed using ordinary one-way ANOVA. Data in FIG. 2F was also subjected to Tukey's multiple comparison test with a single pooled variance to compare each column to every other column.

EC50 of rhPD-1-Fc for PD-L1 CAR NK cells was determined using the GraphPad Prism analysis “Nonlinear Regression (curve fit): [Agonist] vs. response—Variable slope (four parameters)” as described here: <<https://www.graphpad.com/guides/prism/7/curve-fitting/index.htm?REG_DR_stim_variable_2.htm>>

### Results

### PD-1 is a Selective Marker of Human T_{FH} Cells

The PDCD1 gene encoding PD-1 is expressed at low to intermediate levels on several types of leukocytes, but is expressed at higher levels by bona fide T_{FH} cells. Flow cytometer-acquired cell-surface expression of PD-1 on resting, non-inflamed human tonsil mononuclear revealed that electronically gated T_{FH} cells (CD3⁺ CD4⁺ CXCR5⁺ ICOS⁺) exhibit the highest degree of PD-1 expression relative to other major tonsillar leukocyte subsets (FIG. 1A). The geometric mean fluorescence intensity (MFI) of PD-1 expression on T_{FH} cells exceeded that of regulatory T cells (Treg, CD3⁺ CD4⁺ CD25⁺ FoxP3⁺); naïve (CD45RA⁺ CD45RO^{neg}) or memory (CD45RA^{neg} CD45RO⁺) T cells (CD3⁺, CD4⁺ or CD8⁺); immature (CD21⁺ CD38^{hi} CD27^{neg} IgM⁺ IgD^{neg}), mature (CD27⁺ IgD⁺ IgM⁺), memory (CD21⁺ CD27⁺ CD38^{neg}), and follicular (CD21⁺ CD38^{lo} CD27^{neg} IgD⁺ IgM^{neg}) B cells (CD3^{neg} CD19⁺ CD20⁺); plasmablasts (CD3^{neg} CD19⁺ CD27⁺ CD138⁺ CD38^{hi}); NK (CD45⁺ CD3^{neg} CD56⁺) or natural killer T (NKT; CD45⁺ CD3⁺ CD56⁺) cells; classical (CD16^{neg}) and non-classical (CD16⁺) monocytes (CD45⁺ CD3^{neg} CD19^{neg} CD14⁺); and dendritic cells (CD45⁺ CD3^{neg} CD19^{neg} CD1c⁺ HLA-DR⁺) in both human tonsil (FIG. 1B) and peripheral blood mononuclear cells (PBMC) (FIG. 1C). PD-1 expression on tonsillar T_{FH} cells was only surpassed by the closely-related, albeit >1,600-fold less abundant (data not shown) follicular regulatory T cell (T_{FR}, CD3⁺ CD4⁺ CXCR5⁺ ICOS⁺ CD25⁺ Foxp3⁺) population (FIG. 1B). Thus, T_{FH} cells demonstrated roughly 6- to 600-fold higher PD-1 expression (by MFI) than most other leukocytes.

### PD-L1-Based CAR Generation and Expression on NK Cells

Without being bound by theory, we hypothesize that the lower affinity of PD-L1 for PD-1, relative to the scFv of an anti-PD-1 antibody, would permit more selective targeting of PD-1^{high T}_{FH} as compared to PD-1^{low} cells (PD-1^{low} cells are cells that express lower amounts of PD-1 compared to T_{FH} cells, such as bystander cells). We find here that selective targeting of PD-1^{high} T_{FH} cells may be achieved by optimizing the affinity of a CAR (e.g., via PD-L1 or a fragment or variant thereof) to limit its activation by PD-1^{low} cells.

We cloned the extracellular sequence of human PD-L1 (a.a. 19-238) upstream of conventional CAR components (FIGS. 1D and 1E), into a lentiviral plasmid. Empty and PD-L1-CAR-containing plasmids were packaged in vesicular stomatitis virus glycoprotein-pseudotyped viral particles and subsequently used to transduce the human NK cell-line, NK-92. Based on fluorescent reporter expression, transduction efficiency in NK-92 cells was 6.8% and 1.3% for the empty and CAR-encoding lentiviral vectors, respectively, as measured by flow cytometry (FIG. 1F). After sorting for fluorescent reporter-positive cells, CAR transgene mRNA and surface PD-L1 were detected on CAR-transduced but not empty vector-transduced NK-92 (FIGS. 1G and 1H).

### PD-L1 CAR NK Cells are Activated by Plate-Bound Ligands

CAR NK-92 cells in short-term culture with either plate-bound antibody specific for PD-L1 (α-PD-L1) or recombinant human PD-1-Fc fusion protein (rhPD-1-Fc) triggered degranulation, as measured by surface exposure of CD107a (FIG. 2A). This response was not observed in control NK-92 cells. Neither CAR nor control NK-92 responded to IgG-Fc fusion protein or immunoglobulin isotype (negative controls), while both CAR and control NK-92 responded robustly to stimulation with (positive control) phorbol myristate acetate (PMA) and ionomycin (FIG. 2A). Stimulation of CAR NK-92 over increasing concentrations of plate-bound rhPD-1-Fc (FIG. 2B) revealed a response curve with an affinity of PD-L1 CAR NK-92 for rhPD-1-Fc with a calculated EC50 of 0.61 μg/mL. These experimental findings establish that our PD-L1-based CAR construct was functional and responsive to ligands that bind PD-L1.

### PD-L1 CAR NK Cells Respond to Cell-Associated PD-1 Via Degranulation and Killing

The Drosophila melanogaster-derived S2 cell line lacks expression of relevant activating or inhibitory receptors for human NK cells, and therefore elicits no functional NK-cell response. Consistent with this, NK-92 cells did not degranulate when co-cultured with S2 cells, regardless of CAR expression (FIG. 2C). In contrast, CAR NK-92 but not control NK-92 degranulated (FIG. 2C) during co-culture with S2 cells engineered to express surface human PD-1 (FIG. 2G). Moreover, CAR NK-92 cells degranulated 34% more (FIG. 2D) during co-culture with sorted “PD-1^{Hi}” S2 cells (FIG. 2G) than CAR NK-92 cell co-cultured with “PD-1^{Lo}” S2 cells. Similarly, Raji human B lymphoma cells engineered to express high levels of human PD-1 (FIG. 2H) triggered 2.4-fold more degranulation of CAR NK-92 relative to degranulation in response to wild-type Raji cells (FIG. 2E). Furthermore, PD-1+Raji cells cultured in the presence of CAR NK-92, but not those cultured with control NK-92, exhibited uptake of propidium iodide (PI) as a measure of loss of viability (FIG. 2F). Thus, CAR NK-92 recognize and respond to cell-surface PD-1 on otherwise NK-cell refractory target cells.

### PD-L1 CAR NK Cells Selectively Kill Tonsil T_{FH} Cells In Vitro

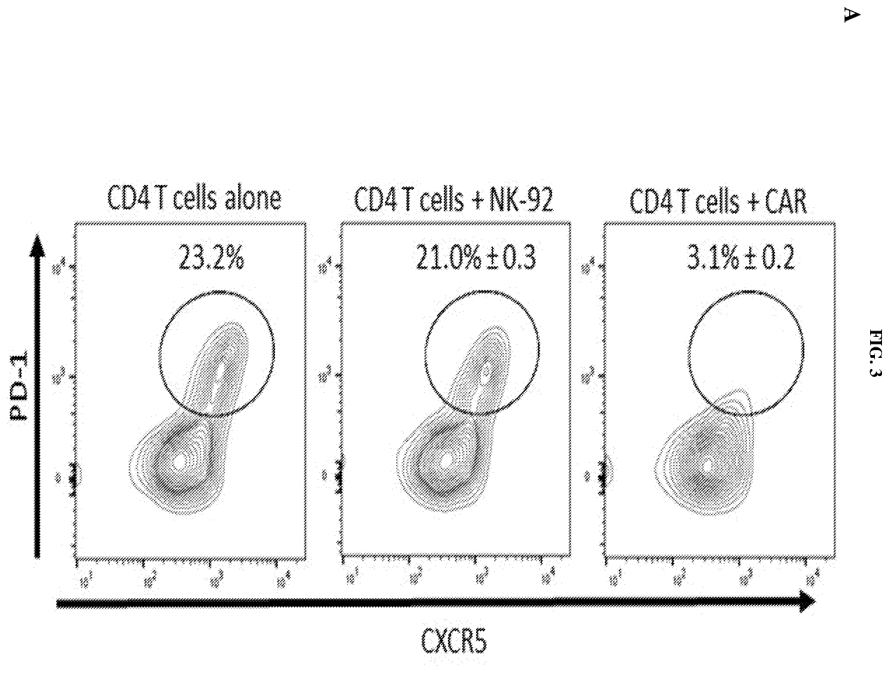

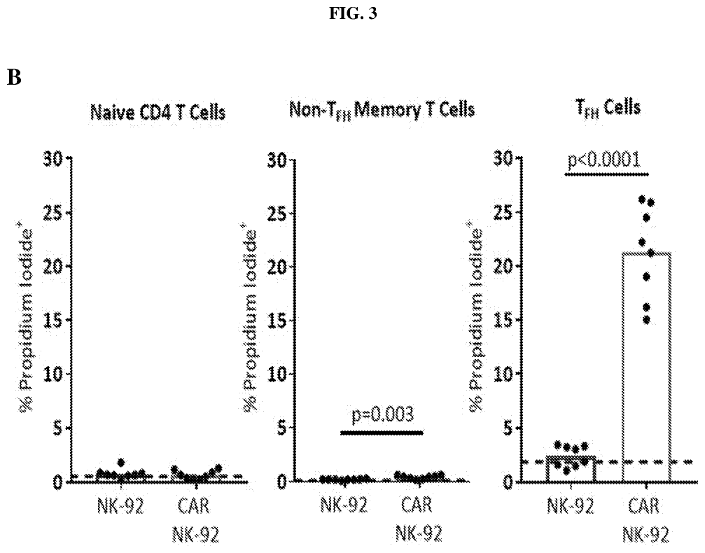

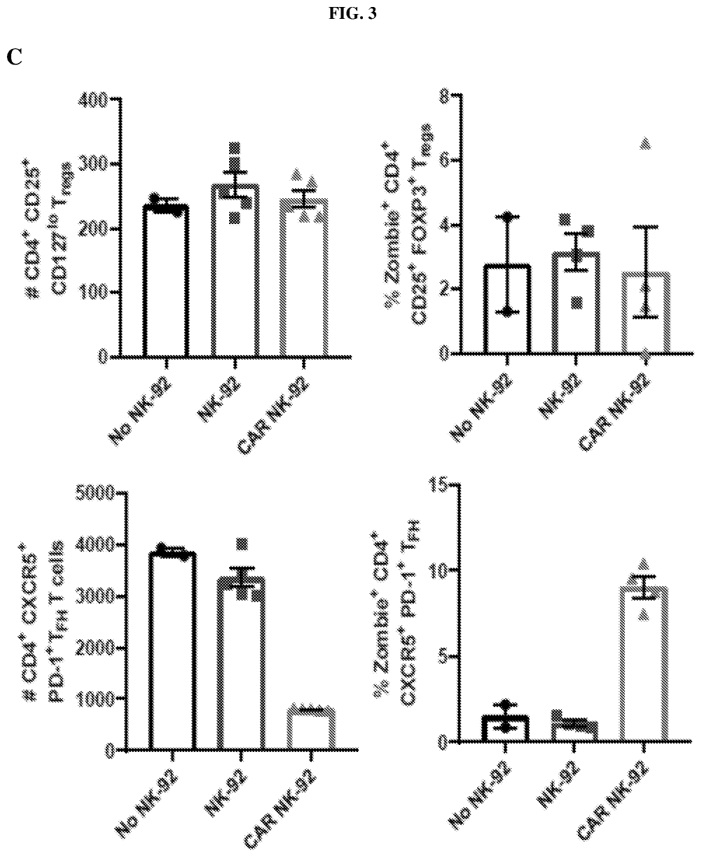

To test the function of PD-L1 CAR NK-92 cells in response to bona fide human T_{FH}, bulk CD4 T cells were magnetically-purified from non-inflamed human tonsil. Co-culture of tonsil CD4 T cells with CAR-expressing NK-92 for 4 hours resulted in greater than a 7-fold reduction in recoverable T_{FH} cells relative to cultures of CD4 T cells alone (FIG. 3A). This was not observed when CD4 T cells were co-cultured with control NK-92 (FIG. 3A). Correspondingly, remaining T_{FH} cells after culture with CAR NK-92 exhibited 9-fold more PI uptake than those cultured with control NK-92 or without added NK cells (FIG. 3B). Non-T_{FH} CD4 memory T cells also exhibited increased loss of viability when cultured with CAR NK-92, though overall PI uptake in this population was lower than that of T_{FH} cells (FIG. 3B). Naïve CD4 T cells demonstrated no such loss of viability over background, despite the addition of either type of NK-92 (FIG. 3B). FIG. 3C shows that the CAR NK-92 cells do not kill Treg cells.

To determine capacity of CAR NK cells to target T_{FH} in a more complex cellular milieu reminiscent of lymphoid follicles, we generated co-cultures of human tonsillar lymphocytes enriched for T_{FH} cells (CD19^{neg} CD3⁺ CD4⁺ CXCR5⁺) and memory B cells (CD3^{neg} CD19⁺ CD27⁺) added in a 1:2 ratio, respectively. Staphylococcal enterotoxin B (SEB), which crosslinks the T-cell receptor with MHCII, was added to these co-cultures to trigger mutual cell proliferation. At an effector (NK-92) to target (T_{FH}) ratio (E:T) of 5:1, CAR NK-92 demonstrated 12-fold more degranulation than control NK-92 (FIG. 4A). Calcein-AM labeled T_{FH} and B cells co-cultured with CAR-expressing but not control NK-92 released calcein into culture supernatant in an effector cell concentration-dependent manner (FIG. 4B). Furthermore, PI staining of target lymphocytes revealed greater loss of viability in T_{FH} cells cultured with CAR NK-92 cells compared to control NK-92 (FIG. 4C). B cells, conversely, exhibited no increased PI uptake following addition of either CAR-expressing or control NK-92 (FIG. 4C). CAR NK cell co-culture with CD4 T cells appears to have an indirect negative effect on downstream proliferation of B cells and IgM production (FIGS. 4D-E). Together, these results demonstrate that PD-L1 CAR-expressing NK-92 promote death of T_{FH} cells (while sparing other lymphocyte populations), thereby indirectly reducing B cell activation and immunoglobulin production.

### Testing of PD-L1 CAR NK in Humanized Mice

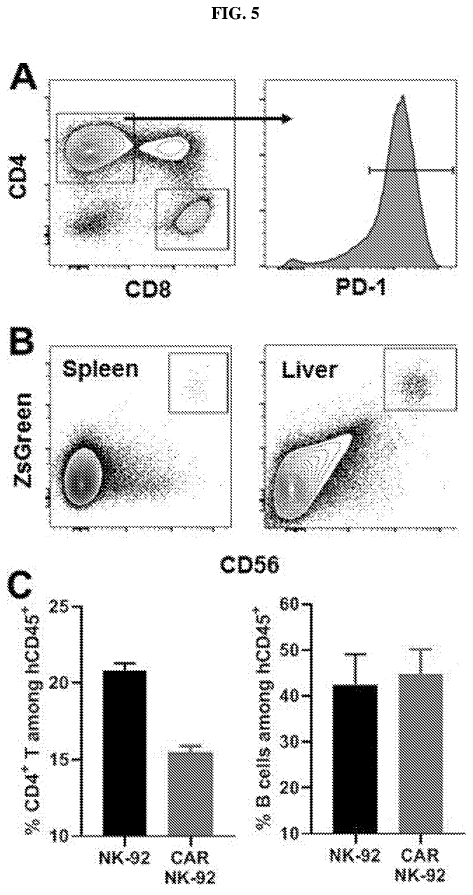

Eight-week-old male and female human IL-3/SCF/GM-CSF transgenic mice on an immune-deficient NOD/LtSz-SCID IL-2RG−/− (NSGS mice) background, bred in-house, were conditioned via i.p. injections of 30 mg/kg busulfan (Sigma Aldrich) and kept on doxycycline chow throughout experiments. Twenty-four hours post-conditioning, mice were humanized via i.v. injection of 10-15 million density-gradient purified (RBC-depleted), T cell-depleted (magnetic column; Miltenyi Biotec) cord blood leukocytes. After 4-5 weeks of reconstitution, mice were given an intraperitoneal injection of 0.5 mL pristane (Sigma Aldrich). Six weeks after injection of pristane, mice (n=3) exhibit an expansion of CD4 T cells that are uniformly PD-1^{high} (FIG. 5A). These mice also have expanded populations of B cells, hyperactive T and B cells, elevated titers of autoantibodies, increased proinflammatory cytokine expression, and progressive SLE disease symptoms including lymphopenia, pulmonary serositis, and lupus nephritis. A bolus of 10⁷ CAR-expressing NK92 cells was introduced into these mice by intravenous injection. Twenty-four hours after injection, we measured ZsGreen⁺ CD56⁺ donor NK92 cells in spleen and liver by flow cytometry (FIG. 5B). In a separate group of pristane-treated humanized mice (n=2), we intravenously injected either 5×10⁶ irradiated (10 Gy γ-ray) control or CAR NK92 three times over 10 days. The recovery of human CD4⁺CD45⁺ T cells and CD19⁺CD45⁺ B cells was evaluated in the spleen of treated mice (FIG. 5C).

### Discussion

In these examples, we designed a PD-L1-based CAR that permits NK cells to selectively and efficiently target T_{FH} cells based upon their markedly elevated expression of PD-1 relative to other leukocytes in human blood or tonsil. Expression of this CAR construct on human NK-92 cells conferred a capacity for cytolytic degranulation in response to PD-1 presented on the surface of tissue-culture plates, insect cells, Raji tumor cells, or bona fide human T_{FH}. CAR-expressing NK cells selectively eliminated T_{FH} but not B cells or naïve T cells during in vitro co-cultures. Thus, our results demonstrate that the PD-L1 CAR can confer a substantial degree of specificity for target cells based on PD-1 expression levels.

We also conclude that PD-1+CD4 T cells in a humanized mouse model of pristane-induced SLE can be reduced in frequency by administration of PD-L1 CAR NK92 cells.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A chimeric polypeptide, wherein the chimeric polypeptide comprises (a) an extracellular segment comprising PD-L1 (Programmed Death-Ligand 1) or a portion of PD-L1, or one or more modifications thereof, (b) a transmembrane segment, and (c) an intracellular segment comprising a first signaling polypeptide and optionally one or more second signaling polypeptides.

2. The chimeric polypeptide of claim 1, wherein the PD-L1 is a mammalian PD-L1, a mouse PD-L1, a dog PD-L1, a cat PD-L1, a rat PD-L1, a pig PD-L1, a woodchuck PD-L1, a cow PD-L1, a monkey PD-L1, a cynomolgus monkey PD-L1, a primate PD-L1, a human PD-L1, or SEQ ID NO:38.

3. The chimeric polypeptide of claim 1, wherein the extracellular segment comprises SEQ ID NO: 14 or SEQ ID NO: 15, or one or more modifications thereof.

4. The chimeric polypeptide of claim 1, wherein the extracellular segment further comprises one or more spacer sequences, where (a) each of the one or more spacer sequences comprises from 5 amino acids to about 1000 amino acids, and/or (b) the one or more of the spacer sequences comprises CD8α, a portion of CD8α, a hinge CD8α sequence, a leader CD8α sequence, CD8β, a portion of CD8β, CD4, a portion of CD4, CD28, or a portion of CD28, or one or more modifications thereof, or combinations thereof.

5. (canceled)

6. The chimeric polypeptide of claim 1, wherein the extracellular segment comprises one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or SEQ ID NO:15, or a portion thereof, or one or more modifications thereof.

7. The chimeric polypeptide of claim 1, wherein the transmembrane segment comprises SEQ ID NO:16 or SEQ ID NO:17, or portions thereof, or one or more modifications thereof.

8. The chimeric polypeptide of claim 1, wherein the first signaling polypeptide is CD3ζ, FcRγ, FcRβ, CD3γ, CD3δ, CD3ε, CD5, CD22, CD79a, CD79b, or CD66d, or portions thereof, or one or more modifications thereof, and/or wherein the first signaling polypeptide comprises SEQ ID NO: 18, SEQ ID NO: 19, or SEQ ID NO:20, or one or more modifications thereof.

9. (canceled)

10. The chimeric polypeptide of claim 1, wherein the intracellular segment comprises a second signaling polypeptide and where (a) the second signaling polypeptide is CD2, CD4, CD5, CD8α, CD8β, CD28, CD134, CD137, ICOS, or CD154, or portions thereof, or one or more modifications thereof, and/or (b) the second signaling polypeptide comprises SEQ ID NO:21 or SEQ ID NO:22, or one or more modifications thereof.

11. (canceled)

12. The chimeric polypeptide of claim 1, wherein the intracellular segment comprises SEQ ID NO:18, SEQ ID NO:19, SEQ ID NO:20, SEQ ID NO:21, SEQ ID NO:22, SEQ ID NO:23, or SEQ ID NO:24, or one or more modifications thereof.

13. The chimeric polypeptide of claim 1, wherein the chimeric polypeptide comprises one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO: 18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, or SEQ ID NO:38, or one or more modifications thereof.

14. The chimeric polypeptide of claim 1, wherein the chimeric polypeptide comprises SEQ ID NO:25, SEQ ID NO:26, SEQ ID NO:27, SEQ ID NO:28, SEQ ID NO:29, SEQ ID NO:30, or SEQ ID NO:31, or one or more modifications thereof.

15. A chimeric nucleic acid molecule encoding the chimeric polypeptide of claim 1.

16. The chimeric nucleic acid molecule of claim 15, wherein (a) the chimeric nucleic acid molecule has at least an 80% identity, or at least a 90% identity, to SEQ ID NO:32, or (b) wherein the chimeric nucleic acid molecule encoding the polypeptide is SEQ ID NO: 32.

17.-18. (canceled)

19. The chimeric nucleic acid molecule of claim 15, wherein the chimeric nucleic acid molecule is in a cell, an insect cell, a mammalian cell, a human cell, an sf9 insect cell, a rat cell, a mouse cell, a CHO cell, an immune cell, an immune cell progenitor, a T cell, a T cell progenitor, an NK cell, KHYG1, NK-92, YT, SNK-6, NKL or an NK cell progenitor.

20. The chimeric nucleic acid molecule of claim 15, wherein the chimeric nucleic acid molecule is in an immune cell, an immune cell progenitor, a T cell, a T cell progenitor, an NK cell, KHYG1, NK-92, YT, SNK-6, NKL, or an NK cell progenitor.

21. The chimeric nucleic acid molecule of claim 15, wherein the chimeric nucleic acid molecule is in a cell isolated from an animal, a mammal, a primate, or a human, and/or wherein the chimeric nucleic acid molecule is included in a vector, a viral vector, or a plasmid.

22. (canceled)

23. A vector comprising any of the chimeric nucleic acid molecules of claim 15.

24. A cell comprising (a) the chimeric polypeptide of claim 1, (b) a chimeric nucleic acid molecule encoding the chimeric polypeptide of (a), (c) a vector comprising any of the chimeric nucleic acid molecules of (b), or (d) a combination thereof.

25. The cell of claim 24, wherein the cell is an immune cell, a stem cell, a mammalian cell, a primate cell, or a human cell.

26. The cell of claim 24, wherein the cell is autologous or allogeneic, and/or wherein the cell is a T cell, a CD8-positive T cell, a CD4-positive T cell, a regulatory T cell, a cytotoxic T cell, a tumor infiltrating lymphocyte, an NK cell, an KHYG1 cell, an NK-92 cell, a YT cell, an SNK-6 cell, or an NKL cell.

27.-28. (canceled)

29. A composition comprising (a) the chimeric polypeptide of claim 1, (b) a chimeric nucleic acid molecule encoding the chimeric polypeptide of (a), (c) a vector comprising any of the chimeric nucleic acid molecules of (b), (d) a cell comprising one or more of (a), (b), or (c), or (e) a combination thereof.

30. The composition of claim 29, wherein the amount of the chimeric polypeptide, the chimeric nucleic acid molecule, the chimeric vector, or the cell is from about 0.0001% (by weight total composition) to about 99%.

31. A pharmaceutical composition comprising (a) the chimeric polypeptide of claim 1, (b) a chimeric nucleic acid molecule encoding the chimeric polypeptide of (a), (c) a vector comprising any of the chimeric nucleic acid molecules of (b), (d) a cell comprising one or more of (a), (b), or (c), or (e) a combination thereof.

32. The pharmaceutical composition of claim 31, wherein the amount of the chimeric polypeptide, the chimeric nucleic acid molecule, the chimeric vector, or the cell is from about 0.0001% (by weight total composition) to about 50%.

33. A method of producing a second cell of comprising adding to a first cell (a) the chimeric nucleic acid molecule of claim 15, (b) a vector comprising any of the chimeric nucleic acid molecules of (a), or (c) a combination thereof.

34. A method of producing a second cell comprising
(a) isolating a first cell,
(b) adding to the isolated first cell, (1) the chimeric nucleic acid molecule of claim 15, (2) a vector comprising any of the chimeric nucleic acid molecules of (1), or (3) a combination thereof, to result in the second cell, and
(c) optionally recovering and/or expanding the resulting second cell from step (b).

35. The cell of claim 33, wherein the first cell is an immune cell, a stem cell, a mammalian cell, a primate cell, or a human cell, and/or wherein the first cell is autologous or allogeneic.

36. (canceled)

37. The cell of claim 33, wherein the first cell is a T cell, a CD8-positive T cell, a CD4-positive T cell, a regulatory T cell, a cytotoxic T cell, or a tumor infiltrating lymphocyte.

38. The cell of claim 33, wherein the first cell is an NK cell, an KHYG1 cell, an NK-92 cell, a YT cell, an SNK-6 cell, or an NKL cell.

39. A method for treating an animal with a disease comprising administering (a) the cell of claim 24, (b) a composition comprising the cell of (a), (b) a pharmaceutical composition comprising the cell of (a), or (c) a combination thereof.

40. The method of claim 39, wherein the disease is autoimmune disease, allergies, asthma, Sjögren's syndrome, juvenile dermatomyositis, multiple sclerosis, type-1 diabetes mellitus, rheumatoid arthritis, systemic lupus erythematosus (SLE), cancer, blood cancer, solid tumor, leukemia, lymphoma, myeloma, breast cancer, ovarian cancer, glioblastoma, osteosarcoma, medulloblastoma, inflammatory disease, eczema, hepatitis, or an infectious disease.

41. The method of claim 39, wherein the disease is autoimmune disease, Sjögren's syndrome, juvenile dermatomyositis, multiple sclerosis, type-1 diabetes mellitus, rheumatoid arthritis, systemic lupus erythematosus (SLE), or cancer.

42. The method of claim 39, wherein the animal is a mammal, primate, human, mouse, or rat, and/or wherein the animal is in need of treatment.

43. The method of claim 39, wherein the administration comprises parenteral administration, a mucosal administration, intravenous administration, subcutaneous administration, topical administration, intradermal administration, oral administration, sublingual administration, intranasal administration, or intramuscular administration.

44. (canceled)


SEQUENCE LISTING (50 sequences)

SEQ ID NO: 1 - protein, 304 aa, Artificial Sequence
  CD8 alpha  leader, PD-L1 signal, PD-L1 extracellular, CD8 alpha hinge
MALPVTALLLPLALLLHAARPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACD

SEQ ID NO: 2 - protein, 283 aa, Artificial Sequence
  PD-L1 signal, PD-L1 extracellular, CD8alpha hinge
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACD

SEQ ID NO: 3 - protein, 265 aa, Artificial Sequence
  PD-L1 extracellular, CD8alpha hinge
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACD

SEQ ID NO: 4 - protein, 50 aa, Artificial Sequence
  ADYKR, CD8alpha hinge
ADYKRTTTPAPRPPTPAPTIASQPLSLRPEACRPAAGGAVHTRGLDFACD

SEQ ID NO: 5 - protein, 49 aa, Artificial Sequence
  ADYK, CD8alpha hinge
ADYKTTTPAPRPPTPAPTIASQPLSLRPEACRPAAGGAVHTRGLDFACD

SEQ ID NO: 6 - protein, 238 aa, Artificial Sequence
  PD-L1 signal, PD-L1 extracellular
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 7 - protein, 220 aa, Artificial Sequence
  PD-L1 extracellular
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 8 - protein, 26 aa, Artificial Sequence
  CD8alpha leader, ADYKR
MALPVTALLLPLALLLHAARPADYKR

SEQ ID NO: 9 - protein, 25 aa, Artificial Sequence
  CD8 alpha leader, ADYK
MALPVTALLLPLALLLHAARPADYK

SEQ ID NO: 10 - protein, 21 aa, Artificial Sequence
  CD8alpha leader
MALPVTALLLPLALLLHAARP

SEQ ID NO: 11 - protein, 18 aa, Artificial Sequence
  PD-L1 signal
MRIFAVFIFMTYWHLLNA

SEQ ID NO: 12 - protein, 220 aa, Artificial Sequence; identical to SEQ ID NO: 7
  PD-L1 extracellular

SEQ ID NO: 13 - protein, 45 aa, Artificial Sequence
  CD8alpha hinge
TTTPAPRPPTPAPTIASQPLSLRPEACRPAAGGAVHTRGLDFACD

SEQ ID NO: 14 - protein, 5 aa, Artificial Sequence
  CD8 alpha leader, ADYKR only
ADYKR

SEQ ID NO: 15 - protein, 4 aa, Artificial Sequence
  CD8 alpha leader, ADYK only
ADYK

SEQ ID NO: 16 - protein, 29 aa, Artificial Sequence
  CD28 trans 1
FWVLVVVGGVLACYSLLVTVAFIIFWVRS

SEQ ID NO: 17 - protein, 27 aa, Artificial Sequence
  CD trans 2
FWVLVVVGGVLACYSLLVTVAFIIFWV

SEQ ID NO: 18 - protein, 151 aa, Artificial Sequence
  CD28 intracellular, CD3zeta
KRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 112 aa, Artificial Sequence
  CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 39 aa, Artificial Sequence
  CD28 intracellular
KRSRLLHSDYMNMTPRRPGPTRKHYQPYAPPRDFAAYRS

SEQ ID NO: 21 - protein, 42 aa, Artificial Sequence
  4-1BB
KRGRKKLLYIFKQPFMRPVQTTQEEDGCSCRFPEEEEGGCEL

SEQ ID NO: 22 - protein, 37 aa, Artificial Sequence
  OXO40
RRDQRLPPDAHKPPGGGSFRTPIQEEQADAHSTLAKI

SEQ ID NO: 23 - protein, 193 aa, Artificial Sequence
  4-1BB + CD28+ CD3zeta
KRGRKKLLYIFKQPFMRPVQTTQEEDGCSCRFPEEEEGGCELKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 188 aa, Artificial Sequence
  OXO40 + CD28+ CD3zeta
RRDQRLPPDAHKPPGGGSFRTPIQEEQADAHSTLAKIKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 484 aa, Artificial Sequence
  Example A - chimeric polypeptide portion
MALPVTALLLPLALLLHAARPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 482 aa, Artificial Sequence
  Example B - chimeric polypeptide portion
MALPVTALLLPLALLLHAARPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACDFWVLVVVGGVLACYSLLVTVAFIIFWVKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 445 aa, Artificial Sequence
  Example C - chimeric polypeptide portion
MALPVTALLLPLALLLHAARPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACDFWVLVVVGGVLACYSLLVTVAFIIFWVRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 524 aa, Artificial Sequence
  Example D - chimeric polypeptide portion
MALPVTALLLPLALLLHAARPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 500 aa, Artificial Sequence
  Example E - chimeric polypeptide portion
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACDFWVLVVVGGVLACYSLLVTVAFIIFWVRSRRDQRLPPDAHKPPGGGSFRTPIQEEQADAHSTLAKIKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 443 aa, Artificial Sequence
  Example F - chimeric polypeptide portion
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTTTPAPRPPTPAPTIASQPLSLRPEACRPAAGGAVHTRGLDFACDFWVLVVVGGVLACYSLLVTVAFIIFWVKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 230 aa, Artificial Sequence
  Example G  - chimeric polypeptide portion
ADYKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 1455 bp, Artificial Sequence
  Example A - NA of chimeric polypeptide portion
atggccttaccagtgaccgccttgctcctgccgctggccttgctgctccacgccgccaggccgatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 33 - DNA, 714 bp, Artificial Sequence
  PD-L1 signaling + extracellular - NA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaagg

SEQ ID NO: 34 - protein, 288 aa, Artificial Sequence
  PD-1 example
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 35 - DNA, 867 bp, Artificial Sequence
  PD-1 example - NA
atgcagatcccgcaagcgccgtggccggtggtctgggctgtcctgcagctcggttggcgccctggttggtttctggacagcccagatcgaccctggaaccccccaacattctcgcctgcactcctcgttgttactgagggcgacaacgcgacttttacgtgcagctttagcaacaccagcgaatcgtttgtgctgaattggtaccggatgagtccgagtaaccagactgataagctcgcagcgtttcccgaagaccgctcccaacctggtcaggattgcaggttccgtgtcacacaactgcccaatggtcgcgactttcacatgagcgtggttcgcgcccgacgtaatgatagcggaacttacttgtgcggtgcaataagcctggcacctaaggctcaaataaaagaatcgctgagggccgaattgagggtaaccgagaggagggctgaggtaccgacggcgcacccatcccccagccctcgacccgccggccaattccagacattggttgtcggagtggtcggcggtttgttgggttcgctcgtgttgttggtgtgggtgctcgcggtgatttgtagccgcgcggcacgaggtaccataggtgctcgtaggaccggacagcctttgaaggaggacccgtccgccgtgccggtcttttcggtggactatggagagctcgatttccaatggcgtgaaaaaactccagagcctccagttccttgtgtcccggagcagacggagtatgcaacgattgtgtttccgtcgggaatgggtacatccagccccgcccgacgcggctccgcggacggtccgaggagtgcccaacctctgcggcccgaagacggacactgctcgtggcctctgtaa

SEQ ID NO: 36 - DNA, 9677 bp, Artificial Sequence
  chimeric polypeptide in pLVX-IRES-ZsGreen plasmid
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcctcgagactagtctcgaggaattcatggccttaccagtgaccgccttgctcctgccgctggccttgctgctccacgccgccaggccgatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgactctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 37 - DNA, 6199 bp, Artificial Sequence
  pAc/V5-His-PD1 plasmid
aattcatgcagatcccgcaagcgccgtggccggtggtctgggctgtcctgcagctcggttggcgccctggttggtttctggacagcccagatcgaccctggaaccccccaacattctcgcctgcactcctcgttgttactgagggcgacaacgcgacttttacgtgcagctttagcaacaccagcgaatcgtttgtgctgaattggtaccggatgagtccgagtaaccagactgataagctcgcagcgtttcccgaagaccgctcccaacctggtcaggattgcaggttccgtgtcacacaactgcccaatggtcgcgactttcacatgagcgtggttcgcgcccgacgtaatgatagcggaacttacttgtgcggtgcaataagcctggcacctaaggctcaaataaaagaatcgctgagggccgaattgagggtaaccgagaggagggctgaggtaccgacggcgcacccatcccccagccctcgacccgccggccaattccagacattggttgtcggagtggtcggcggtttgttgggttcgctcgtgttgttggtgtgggtgctcgcggtgatttgtagccgcgcggcacgaggtaccataggtgctcgtaggaccggacagcctttgaaggaggacccgtccgccgtgccggtcttttcggtggactatggagagctcgatttccaatggcgtgaaaaaactccagagcctccagttccttgtgtcccggagcagacggagtatgcaacgattgtgtttccgtcgggaatgggtacatccagccccgcccgacgcggctccgcggacggtccgaggagtgcccaacctctgcggcccgaagacggacactgctcgtggcctctgtaagcggccgcctcgagtctagagggcccttcgaaggtaagcctatccctaaccctctcctcggtctcgattctacgcgtaccggtcatcatcaccatcaccattgagtttaaacccgctgatcagcctcgactgtgccttctaaggcctgagctcgctgatcagcctcgatcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaagggcagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctatattctaaaaacacaaatgatacttctaaaaaaaaatcatgaatggcatcaactctgaatcaaatctttgcagatgcacctacttctcatttccactgtcacatcatttttccagatctcgctgcctgttatgtggcccacaaaccaagacacgttttatggccattaaagctggctgatcgtcgccaaacaccaaatacataatgaatatgtacacattcgagaaagaagcgatcaaagaagcgtcttcgggcggagtaggagaatgcggaggagaaggagaacgagctgatctagtatctctccacaatccaatgccaactgaccaactggccatattcggagcaatttgaagccaatttccatcgcctggcgatcgctccattcttggctatatgtttttcaccgttacccggggccattttcaaagactcgtcggcaagataagattgtgtcactcgctgtctctcttcatttgtcgaagaatgctgaggaatttcgcgatgacgtcggcgagtattttgaagaatgagaataatttgtatttatacgaaaatcagttagtggaattttctacaaaaacatgttatctatagataattttgttgcaaaatatgttgactatgacaaagattgtatgtatatacctttaatgtattctcattttcttatgtatttataatggcaatgatgatactgatgatattttaagatgatgccagaccaaaaggcttgaatttctgcgtcttttgccgaacgcagtgcatgtgcaattgttgttttttggaatattcaattttcggactgtccgctttgatttcagtttcttggcttattcaaaaagcaaagtaaagccaaaaaagcgagatggcaataccaaatgcggcaaaacggtagtggaaggaaaggggtgcggggcagcggaaggaagggtggggcggggcgtggcggggtctgtggctgggcgcgacgtcaccgacgttggagccactcctttgaccatgtgtgcgtgtgtgtattattcgtgtctcgccactcgccggttgtttttttctttttatgctgcgctctctctagcgccatctcgcttacgcatgctcaacgcaccgcatgttgccgtttccttttatgcgtcattttggctcgaaataggcaattatttaaacaaagattagtcaacgaaaacgctaaaataaataagtctacaatatggttacttattgccatgtgtgtgcagccaacgatagcaacaaaagcaacaacacaggtggctttccctctttcactttttgtttgcaagccgcgtgcgagcaagacggcacgaccggcaaacgcaattacgctgacaaagagcagacgaagttttggcgaaaaacatcaaggcgcctgatacgaatgcatttgcaataacaattgcgatatttaatattgtttatgaagctgtttgacttcaaaacacacaaaaaaaaaaataaaacaaattatttgaaagagaattaggaatcggacgcttatcgttagggtaacaacaagaaatgcttactgagtcacagcctctggaaaactgccgcaagccagagagagagagaaaaagagggagagcagcttagaccgcatgtgcttgtgtgtgaggcgtctctctcttcgtctctgttgcgcaaacgcatagactgcactgaaaaaatcgattacctattttttatgaatgaatatttgcactattactattcaaaactattaagatagcaatcacattcaatagccaaatactataccacctgagcgatgcaacgaaatgatcaatttgagcaaaaatgctgcatatttaggacggcatcattatagaaatgcttcttgctgtgtacttttctctcgtctggcagctgtttcgccgttattgttaaaaccggcttaagttaggtgtgttttctacgactagtgaatgccctactagaagatgtgtgttgcacaaaatgtccctggaataaccaatttgaagtgcagatagcagtaaacgtaagctaatatgaatattatttaactgtaatgttttaatatcgctggacattactaataaacccactataaacacatgtacatatgtatgttttggcatacaatgagtagttggggaaaaaatgtgtaaaagcaccgtgaccatcacagcataaagataaccagctgaagtatcgaatatgagtaacccccaaattgaatcacatgccgcaactgataggacccatggaagtacactcttcatggcgatatacaagacacacacaagcacgaacacccagttgcggaggaaattctccgtaaatgaaaacccaatcggcgaacaattcatacccatatatggtaaaagttttgaacgcgacttgagagcggagagcattgcggctgataaggttttagcgctaagcgggctttataaaacgggctgcgggaccagttttcatatcactaccgtttgagttcttgtgctgtgtggatactcctcccgacacaaagccgctccatcagccagcagtcgtctaatccagagaccccggatcggggtacctactagtccagtgtggtgg

SEQ ID NO: 38 - protein, 290 aa, Artificial Sequence
  (6a) - PD-L1 full
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 39 - DNA, 8117 bp, Artificial Sequence
  PB513-PD1 - NA
ggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagacgccaccatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgcgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagccccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgaattcatgcagatcccgcaagcgccgtggccggtggtctgggctgtcctgcagctcggttggcgccctggttggtttctggacagcccagatcgaccctggaaccccccaacattctcgcctgcactcctcgttgttactgagggcgacaacgcgacttttacgtgcagctttagcaacaccagcgaatcgtttgtgctgaattggtaccggatgagtccgagtaaccagactgataagctcgcagcgtttcccgaagaccgctcccaacctggtcaggattgcaggttccgtgtcacacaactgcccaatggtcgcgactttcacatgagcgtggttcgcgcccgacgtaatgatagcggaacttacttgtgcggtgcaataagcctggcacctaaggctcaaataaaagaatcgctgagggccgaattgagggtaaccgagaggagggctgaggtaccgacggcgcacccatcccccagccctcgacccgccggccaattccagacattggttgtcggagtggtcggcggtttgttgggttcgctcgtgttgttggtgtgggtgctcgcggtgatttgtagccgcgcggcacgaggtaccataggtgctcgtaggaccggacagcctttgaaggaggacccgtccgccgtgccggtcttttcggtggactatggagagctcgatttccaatggcgtgaaaaaactccagagcctccagttccttgtgtcccggagcagacggagtatgcaacgattgtgtttccgtcgggaatgggtacatccagccccgcccgacgcggctccgcggacggtccgaggagtgcccaacctctgcggcccgaagacggacactgctcgtggcctctgtaagc

SEQ ID NO: 40 - DNA, 20 bp, Artificial Sequence
  Reverse 1 primer for pLVX
gcacaccggccttattccaa

SEQ ID NO: 41 - DNA, 22 bp, Artificial Sequence
  Reverse 2 primer for pLVX
cattcaacagaccttgcattcc

SEQ ID NO: 42 - DNA, 22 bp, Artificial Sequence
  Forward primer for pLVX
ctactagaggatctatttccgg

SEQ ID NO: 43 - DNA, 18 bp, Artificial Sequence
  Forward primer for pAc/V5
tagaaggcacagtcgagg

SEQ ID NO: 44 - DNA, 20 bp, Artificial Sequence
  Reverse primer for pAc/V5
acacaaagccgctccatcag

SEQ ID NO: 45 - DNA, 22 bp, Artificial Sequence
  Forward primer for PB513
agagctcgtttagtgaaccgtc

SEQ ID NO: 46 - DNA, 18 bp, Artificial Sequence
  Reverse primer for PB513
aactcctcggggactgtg

SEQ ID NO: 47 - DNA, 20 bp, Artificial Sequence
  qPCR, Set A, primer 1
gcaagcattaccagccctat

SEQ ID NO: 48 - DNA, 17 bp, Artificial Sequence
  qPCR, set A, primer 2
ttctggccctgctggta

SEQ ID NO: 49 - DNA, 19 bp, Artificial Sequence
  qPCR, Set B, primer 1
cttaccagtgaccgccttg

SEQ ID NO: 50 - DNA, 21 bp, Artificial Sequence
  qPCR, Set B, primer 2
cttgggaaccgtgacagtaaa